(19) United States
(12) Patent Application Publication
(10) Pub. No.: US 2013/0005728 A1
(43) Pub. Date: Jan. 3, 2013

(54) SUBSTITUTED PYRIMIDINES AS PROSTAGLANDIN D2 RECEPTOR ANTAGONISTS

(75) Inventors: Keith J. HARRIS, Chester, NJ (US); Joacy C. AGUIAR, Newark, NJ (US); Patrick Wai-Kwok SHUM, Flemington, NJ (US); Zhicheng ZHAO, Bridgewater, NJ (US); Gregory B. POLI, Bethlehem, PA (US); Gregory STOKLOSA, Hillsborough, NJ (US); Yong-Mi CHOI-SLEDESKI, Belle Mead, NJ (US); Stephan REILING, Califon, NJ (US); David STEFANY, Succasunna, NJ (US); Charles GARDNER, Flemington, NJ (US)

(73) Assignee: AVENTIS PHARMACEUTICALS INC., Bridgewater, NJ (US)

(21) Appl. No.: 13/610,027

(22) Filed: Sep. 11, 2012

Related U.S. Application Data
(63) Continuation of application No. PCT/US2011/028427, filed on Mar. 15, 2011.
(60) Provisional application No. 61/314,428, filed on Mar. 16, 2010.

(51) Int. Cl. C07D 403/04; A61K 31/5377; A61K 31/506; C07D 401/04; A61P 37/08; A61P 11/06; A61P 11/02; A61P 17/00; A61P 27/02; A61P 27/14; A61P 11/00; A61P 9/10; A61P 3/06; A61P 3/10; C07D 413/04

(52) U.S. Cl. 514/235.8; 544/123; 544/317; 514/252.14; 514/274; 514/255.04

(57) ABSTRACT

The present invention is directed to a substituted pyrimidine compound of formula (I)

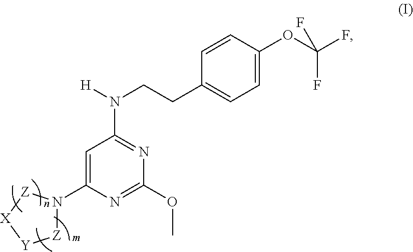

or an enantiomer thereof, or a prodrug or a pharmaceutically acceptable salt thereof, or a pharmaceutical composition comprising such a compound. The invention also includes a method of treatment of a patient by the administration of a pharmaceutically effective amount of such a compound.

### FIELD OF THE INVENTION

The present invention is directed to substituted pyrimidine compounds, their preparation, pharmaceutical compositions containing these compounds, and their pharmaceutical use in the treatment of disease states capable of being modulated by the inhibition of the prostaglandin D2 receptor.

### BACKGROUND OF THE INVENTION

Local allergen challenge in patients with allergic rhinitis, bronchial asthma, allergic conjunctivitis and atopic dermatitis has been shown to result in rapid elevation of prostaglandin D2 “(PGD2)” levels in nasal and bronchial lavage fluids, tears and skin chamber fluids. PGD2 has many inflammatory actions, such as increasing vascular permeability in the conjunctiva and skin, increasing nasal airway resistance, airway narrowing and eosinophil infiltration into the conjunctiva and trachea.

PGD2 is the major cyclooxygenase product of arachidonic acid produced from mast cells on immunological challenge [Lewis, R A, Soter N A, Diamond P T, Austen K F, Oates J A, Roberts L J II, prostaglandin D2 generation after activation of rat and human mast cells with anti-IgE, J. Immunol. 129, 1627-1631, 1982]. Activated mast cells, a major source of PGD2, are one of the key players in driving the allergic response in conditions such as asthma, allergic rhinitis, allergic conjunctivitis, allergic dermatitis and other diseases [Brightling C E, Bradding P, Pavord I D, Wardlaw A J, New Insights into the role of the mast cell in asthma, Clin Exp Allergy 33, 550-556, 2003].

Many of the actions of PGD2 are mediated through its action on the D-type prostaglandin (“DP”) receptor (DP1), a G protein-coupled receptor expressed on epithelium and smooth muscle.

In asthma, the respiratory epithelium has long been recognized as a key source of inflammatory cytokines and chemokines that drive the progression of the disease [Holgate S, Lackie P, Wilson S, Roche W, Davies D, Bronchial Epithelium as a Key Regulator of Airway Allergen Sensitization and Remodelling in Asthma, Am J Respir Crit Care Med. 162, 113-117, 2000]. In an experimental murine model of asthma, the DP receptor is dramatically up-regulated on airway epithelium on antigen challenge [Matsuoka T, Hirata M, Tanaka H, Takahashi Y, Murata T, Kabashima K, Sugimoto Y, Kobayashi T, Ushikubi F, Aze Y, Eguchi N, Urade Y, Yoshida N, Kimura K, Mizoguchi A, Honda Y, Nagai H, Narumiya S, prostaglandin D2 as a mediator of allergic asthma, Science 287, 2013-2017, 2000]. In knockout mice, lacking the DP receptor, there is a marked reduction in airway hyperreactivity and chronic inflammation [Matsuoka T, Hirata M, Tanaka H, Takahashi Y, Murata T, Kabashima K, Sugimoto Y, Kobayashi T, Ushikubi F, Aze Y, Eguchi N, Urade Y, Yoshida N, Kimura K, Mizoguchi A, Honda Y, Nagai H, Narumiya S, Prostaglandin D2 as a mediator of allergic asthma, Science 287, 2013-2017, 2000]; two of the cardinal features of human asthma.

The DP receptor is also thought to be involved in human allergic rhinitis, a frequent allergic disease that is characterized by the symptoms of sneezing, itching, rhinorrhea and nasal congestion. Local administration of PGD2 to the nose causes a dose dependent increase in nasal congestion [Doyle W J, Boehm S, Skoner D P, Physiologic responses to intranasal dose-response challenges with histamine, methacholine, bradykinin, and prostaglandin in adult volunteers with and without nasal allergy, J Allergy Clin Immunol. 86(6 Pt 1), 924-35, 1990].

DP receptor antagonists have been shown to reduce airway inflammation in a guinea pig experimental asthma model [Arimura A, Yasui K, Kishino J, Asanuma F, Hasegawa H, Kakudo S, Ohtani M, Arita H (2001), Prevention of allergic inflammation by a novel prostaglandin receptor antagonist, S-5751, J Pharmacol Exp Ther. 298(2), 411-9, 2001]. PGD2, therefore appears to act on the DP receptor and plays an important role in elicitation of certain key features of allergic asthma.

DP antagonists have been shown to be effective at alleviating the symptoms of allergic rhinitis in multiple species, and more specifically have been shown to inhibit the antigen-induced nasal congestion, the most manifest symptom of allergic rhinitis [Jones, T. R., Savoie, C., Robichaud, A., Sturino, C., Scheigetz, J., Lachance, N., Roy, B., Boyd, M., Abraham, W., Studies with a DP receptor antagonist in sheep and guinea pig models of allergic rhinitis, Am. J. Resp. Crit. Care Med. 167, A218, 2003; and Arimura A, Yasui K, Kishino J, Asanuma F, Hasegawa H, Kakudo S, Ohtani M, Arita H Prevention of allergic inflammation by a novel prostaglandin receptor antagonist, S-5751. J Pharmacol Exp Ther. 298(2), 411-9, 2001].

DP antagonists are also effective in experimental models of allergic conjunctivitis and allergic dermatitis [Arimura A, Yasui K, Kishino J, Asanuma F, Hasegawa H, Kakudo S, Ohtani M, Arita H, Prevention of allergic inflammation by a novel prostaglandin receptor antagonist, S-5751. J Pharmacol Exp Ther. 298(2), 411-9, 2001; and Torisu K, Kobayashi K, Iwahashi M, Nakai Y, Onoda T, Nagase T, Sugimoto I, Okada Y, Matsumoto R, Nanbu F, Ohuchida S, Nakai H, Toda M, Discovery of a new class of potent, selective, and orally active prostaglandin D₂ receptor antagonists, Bioorg. & Med. Chem. 12, 5361-5378, 2004].

Compounds which been identified as DP receptor antagonists are disclosed in PCT patent application WO2006/044732, entitled 2,6-Substituted-4-Monosubstituted Amino-Pyrimidine as Prostaglandin D2 Receptor Antagonists. The compounds of the present invention are all selections within the broad scope of the disclosure of that application.

Macular degeneration is the general term for a disorder in which a part of the retina called the macula deteriorates. Age-related macular degeneration (AMD) is the most common type of macular degeneration. It has been reported that in the United States, AMD is the leading cause of blindness in people older than 55. More than 10 million people in the US are affected by this disease, which includes 23% of people over 90. (www.webmd.com/eye-health/macular-degeneration/macular-degeneration-overview).

The DP1 receptor is highly expressed in the retina of the eye [Boie, Y; Sawyer, D; Slipetta, D M; Metters, K. M.; Abramaovitz, M. Molecular cloning and characterization of the human prostanoid DP receptor, J Biol Chem 270, 18910-18916, 1995]. DP agonists have been shown to cause vasodilation in human retinal microvasculature [Spada, C. S.; Nieves, A. L.; Woodward, D. F. Vascular activities of prostaglandins and selective prostanoid receptor antagonists in human retinal microvessels, Exp. Eye Res. 75, 155-163, 2002].

There are various types of macular degeneration that afflict patients. One type of macular degeneration is “dry” macular degeneration. Dry macular degeneration is an early stage of the disorder in which a pigment is deposited on the macula. The deposition of this pigment may result from aging or thinning of the macular tissues. As a result of this deposition of pigment, loss of central vision may gradually occur. Many times, AMD begins with dry macular degeneration.

Another type of AMD is “wet” macular degeneration. Wet macular degeneration is a neovascular type of degeneration in which blood vessels abnormally grow under the retina and begin to leak. As a result of this leakage, permanent damage occurs to light-sensitive cells of the retina which ultimate causes the death of these cells and thus, blind spots. Unlike dry macular degeneration, in which the vision loss may be minor, the vision loss that occurs in wet macular degeneration can be severe. Indeed, it has been reported that although only 10% of those with AMD suffer from wet macular degeneration, 66% of those with AMD suffering from significant visual loss can directly attribute that loss to wet macular degeneration.

Since the causes for macular degeneration are unknown, there has only been limited success determining the causes for the disorder. Moreover, treatments for macular degeneration have met with only limited success. To date, there is no FDA-approved treatment for dry macular degeneration and nutritional intervention is used to prevent the progression of wet macular degeneration.

Niacin (nicotinic acid) is a drug commonly known for the treatment of hyperlipidemia. The beneficial effects of niacin on the lipid profile include the lowering of plasma levels of cholesterol, triglycerides, free fatty acids and lipoprotein(a) in human. Compared to other lipid-lowering drug, niacin has the special benefit of increasing plasma HDL cholesterol while decreasing LDL and VLDL cholesterol. As a consequence, niacin could potentially be beneficial as an additive therapy to the statins in treating patients with low HDL cholesterol levels.

The major common side effect associated with niacin treatment is flushing. This consists of unpleasant symptoms such as the redness of the skin accompanied by burning sensation, itchiness or irritation mainly affecting upper body and face. These symptoms have a negative impact on patient compliance, and in severe cases, resulted in the discontinuation of niacin treatment. The flushing effect of niacin is transient and lasts for about an hour after taking the drug. In addition, patients develop tolerance to niacin-induced flushing within days while the effects of niacin on improving lipid profile remain stable over time.

The niacin-induced flushing is a result of cutaneous vasodilation (Turenne, S D; Seeman, M; Ross, B. Schizophrenia Research 2001. 50:191-197). Recent studies indicate that the niacin-induced flushing is likely mediated by a G protein-coupled receptor named GPR109A (HM74A in humans, or PUMA-G in mice) (Benyo, Z; Gille, A, et al. The Journal of Clinical Investigation 2005. 115:3634-3640). The mouse ortholog of GPR109A is highly expressed in macrophages and other immune cells (Lorenzen, A; Stannek, C, et al. Biochemical Pharmacology 2002. 64:645-648). Activation of GPR109A by niacin induces the release of prostaglandins, in particular prostaglandin D2 (PGD2), likely from the skin immune cells. PGD2 subsequently acts on its plasma membrane receptor DP (PGD2 receptor) to stimulate the activation of adenylyl cyclase and result in vasodilation/flushing. The involvement of the DP in niacin-induced flushing was further supported by studies using a genetic mouse model lacking the DP receptor (Benyo, Z; Gille, A, et al. The Journal of Clinical Investigation 2005. 115:3634-3640). More recently it was shown that specific DP antagonists inhibited both PGD2 and nicotinic acid-mediated vasodilation in rodents (US Patent Publication No. 20040229844).

### SUMMARY OF THE INVENTION

The present invention is directed to a substituted pyrimidine compound of formula (I)

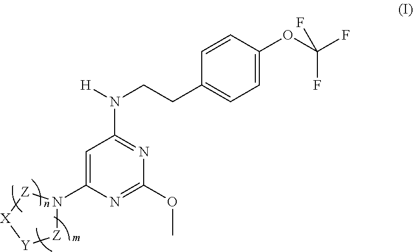

wherein m and n, independently of each other, are selected from the integers 0, 1, 2 or 3;
X and Y, independently of each other, are selected from CR₁R₂, NR₁ or O, wherein X and Y cannot both be O;
or X and Y, taken together with the bond between them, form a phenyl group optionally substituted by one to four R₃ groups;
each Z, independently of each other, is CR₁R₂;
R₁, R₂ and R₃, independently of each other, are selected from the group consisting of H, halogen, aryl, amino, optionally substituted alkyl, optionally substituted alkoxy, and carboxy;
wherein optionally substituted alkyl, may be substituted by one to three of the same or different of halogen, carboxy, cyano, hydroxy, amino or aryl;
wherein optionally substituted alkoxy, may be substituted by one to three of the same or different of halogen, carboxy, cyano, amino or aryl;
wherein each aryl moiety, independently of each other, may be optionally substituted by hydroxy, amino, alkyl, alkoxy, carboxy or alkoxycarbonyl;
provided that a halogen cannot be bonded to an N; and provided the compound is not (1-{2-methoxy-6-[2-(4-trifluoromethoxy-phenyl)-ethylamino]-pyrimidin-4-yl}-piperidin-3-yl)-acetic acid;
or an enantiomer thereof, or a prodrug or a pharmaceutically acceptable salt thereof.

As noted above, the compounds of the present invention are all selections within the broad scope of the disclosure of PCT patent application WO2006/044732. Although many of the compounds disclosed in that application are potent, selective and orally active antagonists of the prostaglandin D2 receptor, it has been found that they increased the amount of CYP3A enzyme. This may negatively affect their potential for development as oral therapies. The selected compounds of the present invention have been found not to have those undesirable levels of CYP3A induction.

Another aspect of the present invention is a pharmaceutical composition comprising, a pharmaceutically effective amount of one or more compounds according to Formula (I) in admixture with a pharmaceutically acceptable carrier.

Another aspect of the present invention is a method of treating a patient suffering from a PGD2-mediated disorder including, but not limited to, allergic disease (such as allergic rhinitis, allergic conjunctivitis, atopic dermatitis, bronchial asthma and food allergy), systemic mastocytosis, disorders accompanied by systemic mast cell activation, anaphylaxis shock, bronchoconstriction, bronchitis, urticaria, eczema, diseases accompanied by itch (such as atopic dermatitis and urticaria), diseases (such as cataract, retinal detachment, inflammation, infection and sleeping disorders) which is generated secondarily as a result of behavior accompanied by itch (such as scratching and beating), inflammation, chronic obstructive pulmonary diseases, ischemic reperfusion injury, cerebrovascular accident, chronic rheumatoid arthritis, pleurisy, ulcerative colitis, macular degeneration, acute macular degeneration, dry macular degeneration and the like by administering to said patient a pharmaceutically effective amount of a compound according to Formula (I).

The present invention further relates to a method for treating or ameliorating macular degeneration in a patient.

Furthermore, in a method of the present invention, administration of a compound to the patient suffering from macular degeneration modulates the activity of an immunocyte in the patient. The activity of numerous types of immunocytes can be modulated in a method of the present invention. Examples of such immunocytes include a natural killer cell (NK cell), a natural killer T cell (NKT cell), a mast cell, a dendritic cell, and granulocyte selected from the group consisting of an eosinophil, a basophil and neutrophil. Naturally, the activity of a combination of these cells can also be modulated in a method of the present invention.

Moreover, a method of the present invention can also be used to treat or ameliorate choroidal neovascularization, which in turn also treats or ameliorates wet macular degeneration in the patient.

Another aspect of the invention relates to a pharmaceutical composition comprising Niacin or a pharmaceutically acceptable salt, or N-oxide thereof, or a nicotinic acid receptor agonist, and a prostaglandin D2 receptor inhibitor, and its pharmaceutical use in the treatment of atherosclerosis, dyslipidemias or diabetes without causing the side effect of flushing. An additional aspect of this invention relates to a pharmaceutical composition comprising a statin, niacin or a pharmaceutically acceptable salt, solvate or N-oxide thereof, or a nicotinic acid receptor agonist, and a prostaglandin D2 receptor inhibitor, and its pharmaceutical use in the treatment of atherosclerosis, dyslipidemias or diabetes without causing the side effect of flushing.

### DETAILED DESCRIPTION OF THE INVENTION

As used above, and throughout the description of the invention, the following terms, unless otherwise indicated, shall be understood to have the following meanings:

“Alkoxy” means alkyl-O—. Exemplary alkoxy includes methoxy, ethoxy, n-propoxy, i-propoxy, n-butoxy, and heptoxy.

“Alkyl” means straight or branched aliphatic hydrocarbon having 1 to about 20 carbon atoms, particularly 1 to about 12 carbon atoms, and more particularly lower alkyl. Branched means that one or more lower alkyl groups such as methyl, ethyl or propyl are attached to a linear alkyl chain. “Lower alkyl” means 1 to 4 carbon atoms in a linear alkyl chain that may be straight or branched.

“Aryl” means an aromatic monocyclic or multicyclic ring system of about 6 to about 14 carbon atoms, particularly 6 to 10 carbon atoms. Exemplary aryl include phenyl and naphthyl.

“Halo” or “halogen” means fluoro, chloro, bromo, or iodo, particularly fluoro or chloro.

“Patient” includes human or other mammal.

“Pharmaceutically acceptable prodrugs” as used herein refers to those prodrugs of the compounds of the present invention which are, within the scope of sound medical judgment, suitable for use in contact with the tissues of patients with undue toxicity, irritation, allergic response commensurate with a reasonable benefit/risk ratio, and effective for their intended use of the compounds of the invention. The term “prodrug” refers to compounds that are transformed in vivo to yield a parent compound of the present invention, for example by hydrolysis in blood. Functional groups that may be rapidly transformed, by metabolic cleavage, in vivo form a class of groups reactive with the carboxyl group of the compounds of this invention. They include, but are not limited to such groups as alkanoyl (such as acetyl, propanoyl, butanoyl, and the like), unsubstituted and substituted aroyl (such as benzoyl and substituted benzoyl), alkoxycarbonyl (such as ethoxycarbonyl), trialkylsilyl (such as trimethyl and triethysilyl), and monoesters formed with dicarboxylic acids (such as succinyl). Because of the ease with which the metabolically cleavable groups of the compounds of this invention are cleaved in vivo, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion is provided in Design of Prodrugs, H. Bundgaard, ed., Elsevier (1985); Methods in Enzymology; K. Widder et al, Ed., Academic Press, 42, 309-396 (1985); A Textbook of Drug Design and Development, Krogsgaard-Larsen and H. Bandaged, ed., Chapter 5; Design and Applications of Prodrugs 113-191 (1991); Advanced Drug Delivery Reviews, H. Bundgard, 8, 1-38, (1992); J. Pharm. Sci., 77, 285 (1988); Chem. Pharm. Bull., N. Nakeya et al, 32, 692 (1984); Pro-drugs as Novel Delivery Systems, T. Higuchi and V. Stella, 14 A.C.S. Symposium Series, and Bioreversible Carriers in Drug Design, E. B. Roche, ed., American Pharmaceutical Association and Pergamon Press, 1987, which are incorporated herein by reference.

“Ester prodrug” means a compound that is convertible in vivo by metabolic means (e.g., by hydrolysis) to a compound of Formula (I). For example an ester of a compound of Formula (I) containing a hydroxy group may be convertible by hydrolysis in vivo to the parent molecule. Alternatively an ester of a compound of Formula (I) containing a carboxy group may be convertible by hydrolysis in vivo to the parent molecule. Exemplary ester prodrugs are methoxy-methyl ester, 1-ethoxycarbonyloxy-ethyl ester, 2-dimethylamino-ethyl ester, ethyl ester and methyl ester.

“Pharmaceutically acceptable salts” refers to the non-toxic, inorganic and organic acid addition salts, and base addition salts, of compounds of the present invention. These salts can be prepared in situ during the final isolation and purification of the compounds.

Suitable esters of compounds of Formula (I) containing a hydroxy group, are for example acetates, citrates, lactates, tartrates, malonates, oxalates, salicylates, propionates, succinates, fumarates, maleates, methylene-bis-b-hydroxynaphthoates, gentisates, isethionates, di-para-toluoyltartrates, methanesulfonates, ethanesulfonates, benzenesulfonates, para-toluenesulfonates, cyclohexylsulfamates and quinates.

Suitable esters of compounds of Formula (I) containing a carboxy group, are for example those described by F. J. Leinweber, Drug Metab. Res., 1987, 18, page 379.

An especially useful class of esters of compounds of Formula (I) containing a hydroxy group, may be formed from acid moieties selected from those described by Bundgaard et. al., J. Med. Chem., 1989, 32, pages 2503-2507, and include substituted (aminomethyl)-benzoates, for example dialkylamino-methylbenzoates in which the two alkyl groups may be joined together and/or interrupted by an oxygen atom or by an optionally substituted nitrogen atom, e.g., an alkylated nitrogen atom, more especially (morpholino-methyl)benzoates, e.g., 3- or 4-(morpholinomethyl)-benzoates, and (4-alkylpiperazin-1-yl)benzoates, e.g., 3- or 4-(4-alkylpiperazin-1-yl)benzoates.

Some of the compounds of the present invention are basic, and such compounds are useful in the form of the free base or in the form of a pharmaceutically acceptable acid addition salt thereof.

Acid addition salts are a more convenient form for use; and in practice, use of the salt form inherently amounts to use of the free base form. The acids which can be used to prepare the acid addition salts include preferably those which produce, when combined with the free base, pharmaceutically acceptable salts, that is, salts whose anions are non-toxic to the patient in pharmaceutical doses of the salts, so that the beneficial inhibitory effects inherent in the free base are not vitiated by side effects ascribable to the anions. Although pharmaceutically acceptable salts of said basic compounds are preferred, all acid addition salts are useful as sources of the free base form even if the particular salt, per se, is desired only as an intermediate product as, for example, when the salt is formed only for purposes of purification, and identification, or when it is used as intermediate in preparing a pharmaceutically acceptable salt by ion exchange procedures. In particular, acid addition salts can be prepared by separately reacting the purified compound in its free base form with a suitable organic or inorganic acid and isolating the salt thus formed. Pharmaceutically acceptable salts within the scope of the invention include those derived from mineral acids and organic acids. Exemplary acid addition salts include the hydrobromide, hydrochloride, sulfate, bisulfate, phosphate, nitrate, acetate, oxalate, valerate, oleate, palmitate, quinates, stearate, laurate, borate, benzoate, lactate tosylate, citrate, maleate, fumarate, succinate, tartrate, naphthylate, mesylate, glucoheptonate, lactiobionate, sulfamates, malonates, salicylates, propionates, methylene-bis-β-hydroxynaphthoates, gentisates, isethionates, di-para-toluoyltartrates, methanesulfonates, ethanesulfonates, benzenesulfonates, para-toluenesulfonates, cyclohexylsulfamates and laurylsulfonate salts. See, for example S. M. Berge, et al., “Pharmaceutical Salts,” J. Pharm. Sci., 66, 1-19 (1977), which is incorporated herein by reference.

Where the compound of the invention is substituted with an acidic moiety, base addition salts may be formed and are simply a more convenient form for use; and in practice, use of the salt form inherently amounts to use of the free acid form. The bases which can be used to prepare the base addition salts include preferably those which produce, when combined with the free acid, pharmaceutically acceptable salts, that is, salts whose cations are non-toxic to the patient in pharmaceutical doses of the salts, so that the beneficial inhibitory effects inherent in the free base are not vitiated by side effects ascribable to the cations. Base addition salts can also be prepared by separately reacting the purified compound in its acid form with a suitable organic or inorganic base derived from alkali and alkaline earth metal salts and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine.

As well as being useful in themselves as active compounds, salts of compounds of the invention are useful for the purposes of purification of the compounds, for example by exploitation of the solubility differences between the salts and the parent compounds, side products and/or starting materials by techniques well known to those skilled in the art.

It will be appreciated that compounds of the present invention may contain asymmetric centers. These asymmetric centers may independently be in either the R or S configuration. It will be apparent to those skilled in the art that certain compounds of the invention may also exhibit geometrical isomerism. It is to be understood that the present invention includes individual geometrical isomers and stereoisomers and mixtures thereof, including racemic mixtures, of compounds of Formula (I) hereinabove. Such isomers can be separated from their mixtures, by the application or adaptation of known methods. Chiral chromatography techniques represent one means for separating isomers from mixtures thereof. Chiral recrystallization techniques may be tried as an alternative means for separating isomers from mixtures thereof. Individual isomeric compounds can also be prepared by employing, where applicable, chiral precursors.

The compounds of present invention and the intermediates and starting materials used in their preparation are named in accordance with IUPAC rules of nomenclature in which the characteristic groups have decreasing priority for citation as the principle group as follows: acids, esters, amides, etc. Alternatively, the compounds are named by AutoNom 4 (Beilstein Information Systems, Inc.).

However, it is understood that, for a particular compound referred to by both a structural Formula and a nomenclature name, if the structural Formula and the nomenclature name are inconsistent with each other, the structural Formula takes the precedence over the nomenclature name.

The compounds of the invention exhibit prostaglandin D2 receptor antagonist activity and are useful a pharmacological acting agents. Accordingly, they are incorporated into pharmaceutical compositions and used in the treatment of patients suffering from certain medical disorders.

Compounds within the scope of the present invention are antagonists of the prostaglandin D2 receptor, according to tests described in the literature and described in pharmacological testing section hereinafter, and which tests results are believed to correlate to pharmacological activity in humans and other mammals. Thus, in a further embodiment, the present invention provides compounds of the invention and compositions containing compounds of the invention for use in the treatment of a patient suffering from, or subject to, conditions, which can be ameliorated by the administration of a PGD2 antagonist. For example, compounds of the present invention could therefore be useful in the treatment of a variety of PGD2-mediated disorders including, but not limited to, allergic disease (such as allergic rhinitis, allergic conjunctivitis, atopic dermatitis, bronchial asthma and food allergy), systemic mastocytosis, disorders accompanied by systemic mast cell activation, anaphylaxis shock, bronchoconstriction, bronchitis, urticaria, eczema, diseases accompanied by itch (such as atopic dermatitis and urticaria), diseases (such as cataract, The present invention further relates to a method for treating or ameliorating macular degeneration, retinal detachment, inflammation, infection and sleeping disorders) which is generated secondarily as a result of behavior accompanied by itch (such as scratching and beating), inflammation, chronic obstructive pulmonary diseases, ischemic reperfusion injury, macular degeneration, acute macular degeneration, cerebrovascular accident, chronic rheumatoid arthritis, pleurisy, ulcerative colitis and the like.

Compounds of the present invention are further useful in treatments involving a combination therapy with:

(i) antihistamines, such as fexofenadine, loratadine, levocetirizine and cetirizine, for the treatment of allergic rhinitis;
(ii) leukotriene antagonists, such as montelukast and zafirlukast, for the treatment of allergic rhinitis, COPD, allergic dermatitis, allergic conjunctivitis, etc., with reference to WO 01/78697;
(iii) beta agonists, such as albuterol, salbuterol and terbutaline, for the treatment of asthma, COPD, allergic dermatitis, allergic conjunctivitis, etc.;
(iv) antihistamines, such as fexofenadine, loratadine, cetirizine and levocetirizine, for the treatment of asthma, COPD, allergic dermatitis, allergic conjunctivitis etc;
(v) PDE4 (phosphodiesterase 4) inhibitors, such as roflumilast and cilomilast, for the treatment of asthma, COPD, allergic dermatitis, allergic conjunctivitis etc; or
(vi) with TP (thromboxane A2 receptor) or CrTh2 (chemoattractant receptor-homologous molecule expressed on Th2 cells) antagonists, such as ramatroban (BAY-u3405), for the treatment of COPD, allergic dermatitis, allergic conjunctivitis, etc.

A special embodiment of the therapeutic methods of the present invention is the treating of allergic rhinitis.

Another special embodiment of the therapeutic methods of the present invention is the treating of bronchial asthma.

Another special embodiment of the therapeutic methods of the present invention is the treating dry macular degeneration.

Another special embodiment of the therapeutic methods of the present invention is the treating wet macular degeneration.

Another special embodiment of the therapeutic methods of the present invention is the amelioration of niacin induced flushing.

According to a further feature of the invention there is provided a method for the treatment of a human or animal patient suffering from, or subject to, conditions which can be ameliorated by the administration of a prostaglandin D2 receptor antagonist, for example conditions as hereinbefore described, which comprises the administration to the patient of an effective amount of compound of the invention or a composition containing a compound of the invention. “Effective amount” is meant to describe an amount of compound of the present invention effective as a prostaglandin D2 receptor antagonist and thus producing the desired therapeutic effect.

References herein to treatment should be understood to include prophylactic therapy as well as treatment of established conditions.

The present invention also includes within its scope pharmaceutical compositions comprising at least one of the compounds of the invention in admixture with a pharmaceutically acceptable carrier.

In practice, the compound of the present invention may be administered in pharmaceutically acceptable dosage form to humans and other animals by topical or systemic administration, including oral, inhalational, rectal, nasal, buccal, intraocular, sublingual, vaginal, colonic, parenteral (including subcutaneous, intramuscular, intravenous, intradermal, intrathecal and epidural), intracisternal and intraperitoneal. It will be appreciated that the preferred route may vary with for example the condition of the recipient.

“Pharmaceutically acceptable dosage forms” refers to dosage forms of the compound of the invention, and includes, for example, tablets, dragées, powders, elixirs, syrups, liquid preparations, including suspensions, sprays, inhalants tablets, lozenges, emulsions, solutions, granules, capsules and suppositories, as well as liquid preparations for injections, including liposome preparations. Techniques and formulations generally may be found in Remington's Pharmaceutical Sciences, Mack Publishing Co., Easton, Pa., latest edition.

A particular aspect of the invention provides for a compound according to the present invention to be administered in the form of a pharmaceutical composition. Pharmaceutical compositions, according to the present invention, comprise compounds of the present invention and pharmaceutically acceptable carriers.

Pharmaceutically acceptable carriers include at least one component selected from the group comprising pharmaceutically acceptable carriers, diluents, coatings, adjuvants, excipients, or vehicles, such as preserving agents, fillers, disintegrating agents, wetting agents, emulsifying agents, emulsion stabilizing agents, suspending agents, isotonic agents, sweetening agents, flavoring agents, perfuming agents, coloring agents, antibacterial agents, antifungal agents, other therapeutic agents, lubricating agents, adsorption delaying or promoting agents, and dispensing agents, depending on the nature of the mode of administration and dosage forms.

Exemplary suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances.

Exemplary antibacterial and antifungal agents for the prevention of the action of microorganisms include parabens, chlorobutanol, phenol, sorbic acid, and the like.

Exemplary isotonic agents include sugars, sodium chloride and the like.

Exemplary adsorption delaying agents to prolong absorption include aluminum monostearate and gelatin.

Exemplary adsorption promoting agents to enhance absorption include dimethyl sulfoxide and related analogs.

Exemplary diluents, solvents, vehicles, solubilizing agents, emulsifiers and emulsion stabilizers, include water, chloroform, sucrose, ethanol, isopropyl alcohol, ethyl carbonate, ethyl acetate, benzyl alcohol, tetrahydrofurfuryl alcohol, benzyl benzoate, polyols, propylene glycol, 1,3-butylene glycol, glycerol, polyethylene glycols, dimethylformamide, Tween® 60, Span® 60, cetostearyl alcohol, myristyl alcohol, glyceryl mono-stearate and sodium lauryl sulfate, fatty acid esters of sorbitan, vegetable oils (such as cottonseed oil, groundnut oil, com germ oil, olive oil, castor oil and sesame oil) and injectable organic esters such as ethyl oleate, and the like, or suitable mixtures of these substances.

Exemplary excipients include lactose, milk sugar, sodium citrate, calcium carbonate and dicalcium phosphate.

Exemplary disintegrating agents include starch, alginic acids and certain complex silicates.

Exemplary lubricants include magnesium stearate, sodium lauryl sulfate, talc, as well as high molecular weight polyethylene glycols.

The choice of pharmaceutical acceptable carrier is generally determined in accordance with the chemical properties of the active compound such as solubility, the particular mode of administration and the provisions to be observed in pharmaceutical practice.

Pharmaceutical compositions of the present invention suitable for oral administration may be presented as discrete units such as a solid dosage form, such as capsules, cachets or tablets each containing a predetermined amount of the active ingredient, or as a powder or granules; as a liquid dosage form such as a solution or a suspension in an aqueous liquid or a non-aqueous liquid, or as an oil-in-water liquid emulsion or a water-in-oil liquid emulsion. The active ingredient may also be presented as a bolus, electuary or paste.

“Solid dosage form” means the dosage form of the compound of the invention is solid form, for example capsules, tablets, pills, powders, dragées or granules. In such solid dosage forms, the compound of the invention is admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alginates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and Na₂CO₃, (e) solution retarders, as for example paraffin, (f) absorption accelerators, as for example, quaternary ammonium compounds, (g) wetting agents, as for example, cetyl alcohol and glycerol monostearate, (h) adsorbents, as for example, kaolin and bentonite, (i) lubricants, as for example, talc, calcium stearate, magnesium stearate, solid polyethylene glycols, sodium lauryl sulfate, (j) opacifying agents, (k) buffering agents, and agents which release the compound(s) of the invention in a certain part of the intestinal tract in a delayed manner.

A tablet may be made by compression or molding, optionally with one or more accessory ingredients. Compressed tables may be prepared by compressing in a suitable machine the active ingredient in a free-flowing form such as a powder or granules, optionally mixed with a binder, lubricant, inert diluent, preservative, surface active or dispersing agent. Excipients such as lactose, sodium citrate, calcium carbonate, dicalcium phosphate and disintegrating agents such as starch, alginic acids and certain complex silicates combined with lubricants such as magnesium stearate, sodium lauryl sulfate and talc may be used. A mixture of the powdered compounds moistened with an inert liquid diluent may be molded in a suitable machine to make molded tablets. The tablets may optionally be coated or scored and may be formulated so as to provide slow or controlled release of the active ingredient therein.

Solid compositions may also be employed as fillers in soft and hard-filled gelatin capsules using such excipients as lactose or milk sugar as well as high molecular weight polyethylene glycols, and the like.

If desired, and for more effective distribution, the compounds can be microencapsulated in, or attached to, a slow release or targeted delivery systems such as a biocompatible, biodegradable polymer matrices (e.g., poly(d,l-lactide co-glycolide)), liposomes, and microspheres and subcutaneously or intramuscularly injected by a technique called subcutaneous or intramuscular depot to provide continuous slow release of the compound(s) for a period of 2 weeks or longer. The compounds may be sterilized, for example, by filtration through a bacteria-retaining filter, or by incorporating sterilizing agents in the form of sterile solid compositions that can be dissolved in sterile water, or some other sterile injectable medium immediately before use.

“Liquid dosage form” means the dose of the active compound to be administered to the patient is in liquid form, for, example, pharmaceutically acceptable emulsions, solutions, suspensions, syrups and elixirs. In addition to the active compounds, the liquid dosage forms may contain inert diluents commonly used in the art, such solvents, solubilizing agents and emulsifiers.

When aqueous suspensions are used they can contain emulsifying agents or agents which facilitate suspension.

Pharmaceutical compositions suitable for topical administration means formulations that are in a form suitable to be administered topically to a patient. The formulation may be presented as a topical ointment, salves, powders, sprays and inhalants, gels (water or alcohol based), creams, as is generally known in the art, or incorporated into a matrix base for application in a patch, which would allow a controlled release of compound through the transdermal barrier. When formulated in an ointment, the active ingredients may be employed with either a paraffinic or a water-miscible ointment base. Alternatively, the active ingredients may be formulated in a cream with an oil-in-water cream base. Formulations suitable for topical administration in the eye include eye drops wherein the active ingredient is dissolved or suspended in a suitable carrier, especially an aqueous solvent for the active ingredient. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

The oily phase of the emulsion pharmaceutical composition may be constituted from known ingredients in a known manner. While the phase may comprise merely an emulsifier (otherwise known as an emulgent), it desirably comprises a mixture of at least one emulsifier with a fat or an oil or with both a fat and an oil. In a particular embodiment, a hydrophilic emulsifier is included together with a lipophilic emulsifier that acts as a stabilizer. Together, the emulsifier(s) with or without stabilizer(s) make up the emulsifying wax, and the way together with the oil and fat make up the emulsifying ointment base which forms the oily dispersed phase of the cream formulations.

If desired, the aqueous phase of the cream base may include, for example, a least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound that enhances absorption or penetration of the active ingredient through the skin or other affected areas.

The choice of suitable oils or fats for a composition is based on achieving the desired properties. Thus a cream should preferably be a non-greasy, non-staining and washable product with suitable consistency to avoid leakage from tubes or other containers. Straight or branched chain, mono- or dibasic alkyl esters such as di-isopropyl myristate, decyl oleate, isopropyl palmitate, butyl stearate, 2-ethylhexyl palmitate or a blend of branched chain esters known as Crodamol CAP may be used. These may be used alone or in combination depending on the properties required. Alternatively, high melting point lipids such as white soft paraffin and/or liquid paraffin or other mineral oils can be used.

Pharmaceutical compositions suitable for rectal or vaginal administrations means formulations that are in a form suitable to be administered rectally or vaginally to a patient and containing at least one compound of the invention. Suppositories are a particular form for such formulations that can be prepared by mixing the compounds of this invention with suitable non-irritating excipients or carriers such as cocoa butter, polyethylene glycol or a suppository wax, which are solid at ordinary temperatures but liquid at body temperature and therefore, melt in the rectum or vaginal cavity and release the active component.

Pharmaceutical composition administered by injection may be by transmuscular, intravenous, intraperitoneal, and/or subcutaneous injection. The compositions of the present invention are formulated in liquid solutions, in particular in physiologically compatible buffers such as Hank's solution or Ringer's solution. In addition, the compositions may be formulated in solid form and redissolved or suspended immediately prior to use. Lyophilized forms are also included. The formulations are sterile and include emulsions, suspensions, aqueous and non-aqueous injection solutions, which may contain suspending agents and thickening agents and anti-oxidants, buffers, bacteriostats and solutes which render the formulation isotonic, and have a suitably adjusted pH, with the blood of the intended recipient.

Pharmaceutical composition of the present invention suitable for nasal or inhalational administration means compositions that are in a form suitable to be administered nasally or by inhalation to a patient. The composition may contain a carrier, in a powder form, having a particle size for example in the range 1 to 500 microns (including particle sizes in a range between 20 and 500 microns in increments of 5 microns such as 30 microns, 35 microns, etc.). Suitable compositions wherein the carrier is a liquid, for administration as for example a nasal spray or as nasal drops, include aqueous or oily solutions of the active ingredient.

Compositions suitable for aerosol administration may be prepared according to conventional methods and may be delivered with other therapeutic agents. Metered dose inhalers are useful for administering compositions according to the invention for an inhalational therapy.

Actual dosage levels of active ingredient(s) in the compositions of the invention may be varied so as to obtain an amount of active ingredient(s) that is (are) effective to obtain a desired therapeutic response for a particular composition and method of administration for a patient. A selected dosage level for any particular patient therefore depends upon a variety of factors including the desired therapeutic effect, on the route of administration, on the desired duration of treatment, the etiology and severity of the disease, the patient's condition, weight, sex, diet and age, the type and potency of each active ingredient, rates of absorption, metabolism and/or excretion and other factors.

Total daily dose of the compounds of this invention administered to a patient in single or divided doses may be in amounts, for example, of from about 0.001 to about 100 mg/kg body weight daily and preferably 0.01 to 10 mg/kg/day. For example, in an adult, the doses are generally from about 0.01 to about 100, preferably about 0.01 to about 10, mg/kg body weight per day by inhalation, from about 0.01 to about 100, preferably 0.1 to 70, more especially 0.5 to 10, mg/kg body weight per day by oral administration, and from about 0.01 to about 50, preferably 0.01 to 10, mg/kg body weight per day by intravenous administration. The percentage of active ingredient in a composition may be varied, though it should constitute a proportion such that a suitable dosage shall be obtained. Dosage unit compositions may contain such amounts of such submultiples thereof as may be used to make up the daily dose. Obviously, several unit dosage forms may be administered at about the same time. A dosage may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. It goes without saying that, for other patients, it will be necessary to prescribe not more than one or two doses per day.

The formulations can be prepared in unit dosage form by any of the methods well known in the art of pharmacy. Such methods include the step of bringing into association the active ingredient with the carrier that constitutes one or more accessory ingredients. In general the formulations are prepared by uniformly and intimately bringing into association the active ingredient with liquid carriers or finely divided solid carriers or both, and then, if necessary, shaping the product.

The formulations may be presented in unit-dose or multi-dose containers, for example sealed ampoules and vials with elastomeric stoppers, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Compounds of the invention may be prepared by the application or adaptation of known methods, by which is meant methods used heretofore or described in the literature, for example those described by R. C. Larock in Comprehensive Organic Transformations, VCH publishers, 1989.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts, Protecting Groups in Organic Synthesis, 3rd edition, John Wiley & Sons, Inc., 1999. Suitable amine protecting groups include sulfonyl (e.g., tosyl), acyl (e.g., benzyloxycarbonyl or t-butoxycarbonyl) and arylalkyl (e.g., benzyl), which may be removed by hydrolysis or hydrogenolysis as appropriate. Other suitable amine protecting groups include trifluoroacetyl [—C(═O)CF₃] which may be removed by base catalyzed hydrolysis, or a solid phase resin bound benzyl group, such as a Merrifield resin bound 2,6-dimethoxybenzyl group (Ellman linker) or a 2,6-dimethoxy-4-[2-(polystyrylmethoxy)ethoxy]benzyl, which may be removed by acid catalyzed hydrolysis, for example with trifluoroacetic acid.

The acid addition salts of the compounds of this invention can be regenerated from the salts by the application or adaptation of known methods. For example, parent compounds of the invention can be regenerated from their acid addition salts by treatment with an alkali, e.g. aqueous sodium bicarbonate solution or aqueous ammonia solution.

Compounds of this invention can be regenerated from their base addition salts by the application or adaptation of known methods. For example, parent compounds of the invention can be regenerated from their base addition salts by treatment with an acid, e.g. hydrochloric acid.

According to a further feature of the invention, base addition salts of the compounds of this invention may be prepared by reaction of the free acid with the appropriate base, by the application or adaptation of known methods. For example, the base addition salts of the compounds of this invention may be prepared either by dissolving the free acid in water or aqueous alcohol solution or other suitable solvents containing the appropriate base and isolating the salt by evaporating the solution, or by reacting the free acid and base in an organic solvent, in which case the salt separates directly or can be obtained by concentration of the solution.

The starting materials and intermediates may be prepared by the application or adaptation of known methods, for example methods as described in the Reference Examples or their obvious chemical equivalents.

High Pressure Liquid Chromatography—Mass Spectrometry (LCMS) experiments to determine retention times (R_{T}) and associated mass ions are performed using the following method.

Mass Spectra (MS) are recorded using a Micromass LCT mass spectrometer. The method is positive electrospray ionization, scanning mass m/z from 100 to 1000. Liquid chromatography is performed on a Hewlett Packard 1100 Series Binary Pump & Degasser; stationary phase: Phenomenex Synergi 2μ Hydro-RP 20×4.0 mm column, mobile phase: A=0.1% formic acid (FA) in water, B=0.1% FA in acetonitrile. Injection volume of 5 μL by CTC Analytical PAL System. Flow is 1 mL/minute. Gradient is 10% B to 90% B in 3 minutes and 90% B to 100% B in 2 minutes. Auxiliary detectors are: Hewlett Packard 1100 Series UV detector, wavelength=220 nm and Sedere SEDEX 75 Evaporative Light Scattering (ELS) detector temperature=46° C., Nitrogen pressure=4 bar.

300 MHz ¹H nuclear magnetic resonance spectra (NMR) are recorded at ambient temperature using a Varian Mercury (300 MHz) spectrometer with an ASW 5 mm probe. In the NMR chemical shifts (6) are expressed ppm relative to tetramethylsilane. Chemical shifts values are indicated in parts per million (ppm) with reference to tetramethylsilane (TMS) as the internal standard.

As used in the examples and preparations that follow, the terms used therein shall have the meanings indicated: “kg” refers to kilograms, “g” refers to grams, “mg” refers to milligrams, “μg” refers to micrograms, “mol” refers to moles, “mmol” refers to millimoles, “M” refers to molar, “mM” refers to millimolar, “μM” refers to micromolar, “nM” refers to nanomolar, “L” refers to liters, “mL” or “ml” refers to milliliters, “4” refers to microliters, “° C.” refers to degrees Celsius, “mp” or “m.p.” refers to melting point, “bp” or “b.p.” refers to boiling point, “mm of Hg” refers to pressure in millimeters of mercury, “cm” refers to centimeters, “nm” refers to nanometers, “abs.” refers to absolute, “conc.” refers to concentrated, “c” refers to concentration in g/mL, “rt” refers to room temperature, “TLC” refers to thin layer chromatography, “HPLC” refers to high performance liquid chromatography, “RP-HPLC” refers to reverse phase high performance liquid chromatography, “i.p.” refers to intraperitoneally, “i.v.” refers to intravenously, “s”=singlet, “d”=doublet; “t”=triplet; “q”=quartet; “m”=multiplet, “dd”=doublet of doublets; “br”=broad, “LC”=liquid chromatograph, “MS”=mass spectrograph, “ESI/MS”=electrospray ionization/mass spectrograph, “Rₜ”=retention time, “M”=molecular ion, “PSI”=pounds per square inch, “DMSO”=Dimethyl sulfoxide, “DMF”=Dimethylformamide, “DCM”=dichloromethane, “HCl”=hydrochloric acid “SPA”=Scintillation Proximity Assay, “ATTC”=American Type Culture Collection, “EtOAc”=ethyl acetate, “THF”=tetrahydrofuran, “MeOH”=methanol, “EtOH”=ethanol, “PBS”=Phosphate Buffered Saline, “TMD”=transmembrane domain, “IBMX”=3-isobutyl-1-methylxanthine, “cAMP”=cyclic adenosine monophosphate.

The present invention is further exemplified, but not limited by, the following illustrative Examples and Intermediates.

### EXAMPLES

### Intermediates

### A. Trifluoro-methanesulfonic acid 2-methoxy-6-trifluoromethanesulfonyl-pyrimidin-4-yl ester

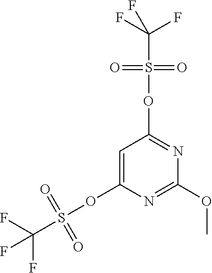 [SMILES: COc1nc(OS(=O)(=O)C(F)(F)F)cc(OS(=O)(=O)C(F)(F)F)n1]

A mixture of 2-methoxy-pyrimidine-4,6-diol (3.0 g, 21.1 mmol) and TEA (11.8 mL, 84.5 mmol) in CH₂Cl₂ (50 mL) was stirred at rt for 1 h. The mixture was cooled in an ice bath, and triflic anhydride (14.3 mL, 84.5 mmol) was added dropwise over a 5 min period. This mixture was stirred at rt for 1 h and concentrated in vacuo. The residue was purified on silica gel with EtOAc/heptane (30%) as eluent to afford the titled product (2.07 g, 24%) as a yellow liquid. ¹H NMR (300 MHz, CDCl₃) δ 6.58 (s, 1H), 4.08 (s, 3H); ¹⁹F NMR (300 MHz, CDCl₃) δ-71.96 (s, 6F); LC Rt 1.12 min; MS 407 (M+1).

### B. 2-(4-trifluoromethoxy-phenyl)-ethylamine hydrochloride

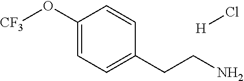

A 500 ml hydrogenation vessel was charged with a solution of (4-trifluoromethoxy-phenyl)-acetonitrile (25.0 g, 124.28 mmol), hydrochloric acid (12N, 25.89 mL, 310.70 mmol) in 200 ml of methyl alcohol and palladium on activated carbon (5 wt %, 13.00 g). The vessel was set in a Parr-shaker apparatus and hydrogenated under 55 PSI of hydrogen overnight (17 hours) at room temperature. The catalyst was removed by filtration over a pad of Celite and the filtrate concentrated under reduced pressure. The solid residue was dissolved in ethyl acetate/dichloromethane (300 mL, 1:1 v/v) and diluted slowly with 200 mL of heptane while stirring vigorously. The precipitated amine salt was collected by filtration to give title compound (25.50 g, 85%). LC/MS: Rt=1.96 minutes, MS m/z=206.

### Trifluoro-methanesulfonic acid 2-methoxy-6-[2-(4-trifluoromethoxy-phenyl)-ethylamino]-pyrimidin-4-yl ester

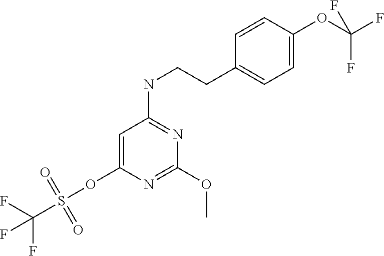 [SMILES: COc1nc(NCCc2ccc(OC(F)(F)F)cc2)cc(OS(=O)(=O)C(F)(F)F)n1]

A solution of trifluoromethanesulfonic acid 2-methoxy-6-trifluoromethanesulfonyl-pyrimidin-4-yl ester (2.07 g, 5.10 mmol), 2-(4-trifluoromethoxy-phenyl)-ethyl amine (1.23 g, 5.10 mmol) and DIEA (1.78 mL, 10.20 mmol) in CH₂Cl₂ (30 mL) was stirred at room temperature for 2 h. The reaction mixture was partitioned between water and CH₂Cl₂. The two layers were separated, and the organic layer was washed with water and brine, dried over Na₂SO₄, filtered, and concentrated in vacuo. The residue was purified on silica gel with EtOAc/heptanes (10-50%) as eluent to afford the titled product (1.83 g, 77%) as a white solid. ¹H NMR (300 MHz, CDCl₃) 6 ¹H NMR (300 MHz, CDCl₃) δ 7.20 (dd, J=8.7, 15.6 Hz, 4H), 5.75 (s, 1H), 3.93 (s, 3H), 3.70 (bs, 2H); ¹⁹F NMR (300 MHz, CDCl₃) δ-57.51 (s, 3F), −72.38 (s, 3F); LC Rt 1.18 min; MS 462 (M+1).

### D. Trifluoro-methanesulfonic acid 6-{tert-butoxycarbonyl-[2-(4-trifluoromethoxy-phenyl)-ethyl]-amino}-2-methoxy-pyrimidin-4-yl ester

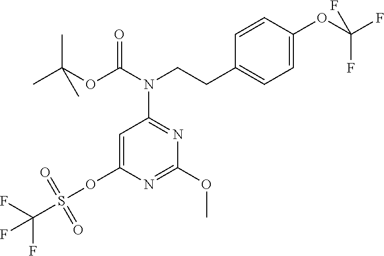 [SMILES: COc1nc(OS(=O)(=O)C(F)(F)F)cc(N(CCc2ccc(OC(F)(F)F)cc2)C(=O)OC(C)(C)C)n1]

A solution of trifluoro-methanesulfonic acid 2-methoxy-6-[2-(4-trifluoromethoxy-phenyl)-ethylamino]-pyrimidin-4-yl ester (1.83 g, 3.96 mmol), di-t-butyl dicarbonate (4 mL, 4.00 mmol, 1.0M in THF) and DMAP (cat) in THF (20 mL) was rt for 3 h. The reaction mixture was partitioned between water and EtOAc. The two layers were separated, and the organic layer was washed with water and brine, dried over Na₂SO₄, filtered, and concentrated in vacuo. The residue was purified on silica gel with EtOAc/heptanes (10-50%) as eluent to afford the titled product (1.96 g, 88%) as a white solid. ¹H NMR (300 MHz, CDCl₃) 6 ¹H NMR (300 MHz, CDCl₃) δ 7.60 (s, 1H), 7.18 (dd, J=8.6, 20.4 Hz, 4H), 4.25 (t, J=7.4 Hz, 2H), 4.02 (s, 3H), 2.92 (t, J=7.6 Hz, 2H), 1.50 (s, 9H); ¹⁹F NMR (300 MHz, CDCl₃) δ-57.53 (s, 3F), −72.48 (s, 3F); LC Rt 1.25 min; MS 562 (M+1).

### E. (6-Chloro-2-methoxy-pyrimidin-4-yl)-[2-(4-trifluoromethoxy-phenyl)-ethyl]-amine

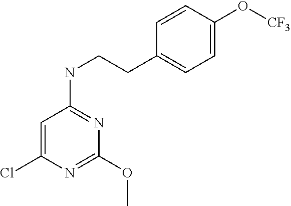 [SMILES: COc1nc(Cl)cc(NCCc2ccc(OC(F)(F)F)cc2)n1]

A suspension of 2-(4-trifluoromethoxy-phenyl)-ethylamine hydrochloride (24.50 g, 101.39 mmol), 4,6-dichloro-2-methoxy-pyrimidine (18.15 g, 101.39 mmol) and sodium hydrogen carbonate (21.29 g, 253.47 mmol) in 300 mL of ethyl alcohol was refluxed at 90° C. for 17 hours. After cooling to room temperature, the reaction was diluted with 450 mL of water and stirring continued for 1.5 hours. The formed precipitate was filtered and air dried to give title compound (34.25 g, 97%). LC/MS: Rt=3.37 minutes, MS m/z=348.

### Example 1

### 4-{2-Methoxy-6-[2-(4-trifluoromethoxy-phenyl)-ethylamino]-pyrimidin-4-yl}-morpholine-2-carboxylic acid trifluoroacetate

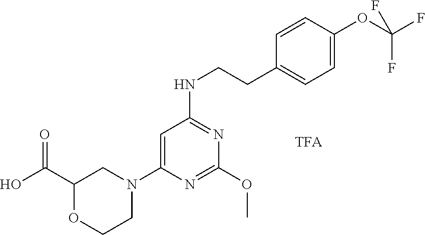 [SMILES: COc1nc(NCCc2ccc(OC(F)(F)F)cc2)cc(N2CCOC(C(=O)O)C2)n1]

### 1A. Trifluoro-methanesulfonic acid 2-methoxy-6-[2-(4-trifluoromethoxy-phenyl)-ethylamino]-pyrimidin-4-yl ester

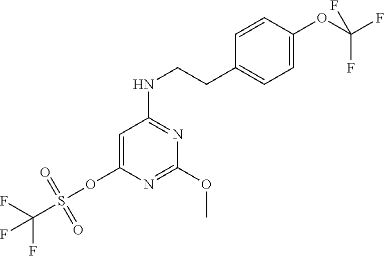 [SMILES: COc1nc(NCCc2ccc(OC(F)(F)F)cc2)cc(OS(=O)(=O)C(F)(F)F)n1]

To a solution of trifluoro-methanesulfonic acid 2-methoxy-6-trifluoromethanesulfonyl-pyrimidin-4-yl ester (105 mg, 0.26 mol) and 2-(4-trifluoromethoxy-phenyl)-ethylamine (53 mg, 0.26 mmol) in CH₂Cl₂ (3 mL) was stirred at rt overnight. More 2-(4-trifluoromethoxy-phenyl)-ethylamine (˜100 mg) was added. After 2 h, LC/MS indicated the reaction was completed. The reaction mixture was concentrated in vacuo, and the crude material was used in the next step without further purification. LC Rt: 1.19 min; MS 462 (M+1).

### 1B. 4-{2-Methoxy-6-[2-(4-trifluoromethoxy-phenyl)-ethylamino]-pyrimidin-4-yl}-morpholine-2-carboxylic acid ethyl ester

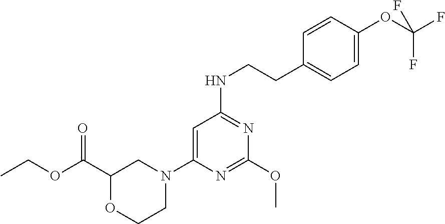 [SMILES: CCOC(=O)C1CN(c2cc(NCCc3ccc(OC(F)(F)F)cc3)nc(OC)n2)CCO1]

A mixture of trifluoromethanesulfonic acid 2-methoxy-6-[2-(4-trifluoromethoxy-phenyl)-ethylamino]-pyrimidin-4-yl ester (from example 1A) and morpholine-2-carboxylic acid ethyl ester (82 mg, 0.51 mmol) in DMF (2 mL) was heated at 95° C. overnight. The solvent was removed in vacuo, and the residue was purified on silica gel with EtOAc/heptanes (33%) as eluent to afford the titled product (79 mg, 65% over 2 steps) as a slightly yellow foam. ¹H NMR (300 MHz, CDCl₃) δ 7.20 (dd, J=8.4, 22.4 Hz, 4H), 5.14 (s, 1H), 4.62 (t, J=6.5 Hz, 1H), 4.36-4.14 (m, 4H), 4.09 (dt, J=3.1, 11.5 Hz, 2H), 3.98-3.88 (m, 1H), 3.87 (s, 3H), 3.69 (dt, J=2.9, 11.2 Hz, 1H), 3.55 (q, J=6.7 Hz, 2H), 3.25-3.11 (m, 2H), 2.91 (t, J=6.6 Hz, 2H), 1.32 (t, J=7.1 Hz, 3H); LC Rt 2.88 min; MS 471 (M+1).

### 1C. 4-{2-Methoxy-6-[2-(4-trifluoromethoxy-phenyl)-ethylamino]-pyrimidin-4-yl}-morpholine-2-carboxylic acid trifluoroacetate

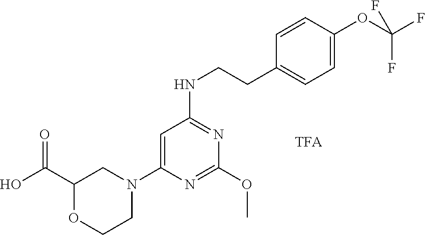 [SMILES: COc1nc(NCCc2ccc(OC(F)(F)F)cc2)cc(N2CCOC(C(=O)O)C2)n1]

A mixture of 4-{2-methoxy-6-[2-(4-trifluoromethoxy-phenyl)-ethylamino]-pyrimidin-4-yl}-morpholine-2-carboxylic acid ethyl ester (70 mg, 0.15 mmol) and NaOH (100 mg, 2.50 mmol) in MeOH (4 mL)/water (2 mL) was heated at 70° C. for 2 h. The solvent was removed in vacuo, and the residue was purified by RP-HPLC to afford the titled product (12.8 mg, 15%) as a white solid. ¹H NMR (300 MHz, CDCl₃) δ 7.34-7.27 (m, 2H), 7.18-7.11 (m, 2H), 4.93 (s, 1H), 4.29-4.02 (m, 4H), 3.98 (s, 3H), 3.75 t, J=10.5 Hz, 1H), 3.47-3.25 (m, 5H), 3.00 (t, J=7.1 Hz, 2H); ¹⁹F NMR (300 MHz, CDCl₃) δ-57.44 (s, 3F), −75.35 (s, 3F); LC Rt 2.53 min; MS 443 (M+1).

hPRP IC₅₀=10 nM

### Example 2

### (4-{2-Methoxy-6-[2-(4-trifluoromethoxy-phenyl)-ethylamino]-pyrimidin-4-yl}-piperazin-1-yl)-acetic acid tristrifluoroacetate

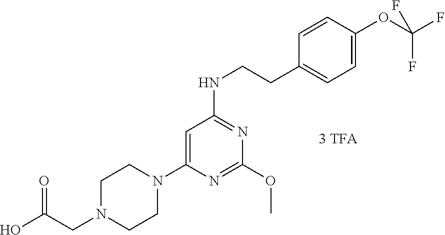 [SMILES: COc1nc(NCCc2ccc(OC(F)(F)F)cc2)cc(N2CCN(CC(=O)O)CC2)n1]

### 2A. [4-(6-{tert-Butoxycarbonyl-[2-(4-trifluoromethoxy-phenyl)-ethyl]-amino}-2-methoxy-pyrimidin-4-yl)-piperazin-1-yl]-acetic acid ethyl ester

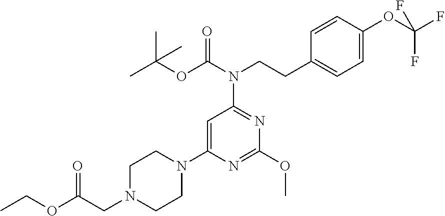 [SMILES: CCOC(=O)CN1CCN(c2cc(N(CCc3ccc(OC(F)(F)F)cc3)C(=O)OC(C)(C)C)nc(OC)n2)CC1]

A solution of trifluoro-methanesulfonic acid 6-{tert-butoxycarbonyl-[2-(4-trifluoromethoxy-phenyl)-ethyl]-amino}-2-methoxy-pyrimidin-4-yl ester (100 mg, 0.18 mmol) and piperazin-1-yl-acetic acid ethyl ester (61 mg, 0.35 mmol) in CH₂Cl₂ (5 mL) was heated at 40° C. overnight. The reaction mixture was concentrated in vacuo, and the residue was purified on silica gel with MeOH/CH₂Cl₂ (2-3%) as eluent to afford the titled product (37 mg, 36%). LC Rt: 3.59 min; MS 584 (M+1).

### 2B. (4-{2-Methoxy-6-[2-(4-trifluoromethoxy-phenyl)-ethylamino]-pyrimidin-4-yl}-piperazin-1-yl)-acetic acid ethyl ester hydrochloride

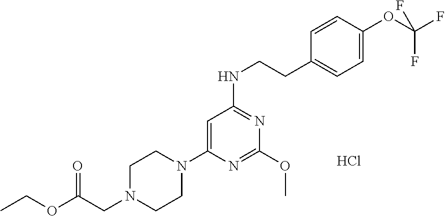 [SMILES: CCOC(=O)CN1CCN(c2cc(NCCc3ccc(OC(F)(F)F)cc3)nc(OC)n2)CC1 ; Cl]

A solution of [4-(6-{tert-butoxycarbonyl-[2-(4-trifluoromethoxy-phenyl)-ethyl]-amino}-2-methoxy-pyrimidin-4-yl)-piperazin-1-yl]-acetic acid ethyl ester (60 mg, 0.10 mmol) in 4M HCl in dioxane (1 mL) was stirred at rt for 2 h. The reaction mixture was concentrated in vacuo, and the crude material was purified on silica gel with MeOH/CH₂Cl₂ (0-50% with NH₄OH) as eluent to afford the titled product (43 mg, 80%) as a yellow foam. ¹⁹F NMR (300 MHz, CDCl₃) δ-57.38 (s, 3F); LC Rt 2.68 min; MS 484 (M+1).

### 2C. (4-{2-Methoxy-6-[2-(4-trifluoromethoxy-phenyl)-ethylamino]-pyrimidin-4-yl}-piperazin-1-yl)-acetic acid tristrifluoroacetate

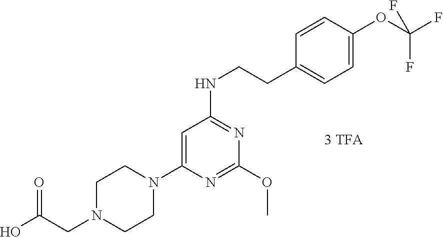 [SMILES: COc1nc(NCCc2ccc(OC(F)(F)F)cc2)cc(N2CCN(CC(=O)O)CC2)n1 ; "3 TFA"]

A mixture of (4-{2-methoxy-6-[2-(4-trifluoromethoxy-phenyl)-ethylamino]-pyrimidin-4-yl}-piperazin-1-yl)-acetic acid ethyl ester hydrochloride (40 mg, 0.08 mmol) and NaOH (100 mg, 2.50 mmol) in MeOH (1.6 mL)/water (0.4 mL) was heated at 70° C. for 2 h. The solvent was removed in vacuo, and the residue was dissolved in water. The pH of the resulting solution was adjusted to seven with 3M HCl and then concentrated to dryness in vacuo. The residue was purified by RP-HPLC to afford the titled product (38 mg, 57%) as a white solid. ¹H NMR (300 MHz, DMSO-d₆) δ 7.33 (dd, J=9.1, 25.8 Hz, 4H), 7.17 (bs, 1H), 5.46 (s, 1H), 5.12-3.83 (m, 7H), 3.79 (s, 3H), 3.55-3.07 (m, 6H), 2.85 (t, J=7.9 Hz, 2H); ¹⁹F NMR (300 MHz, DMSO-d₆) δ-56.37 (s, 3F), −73.96 (s, 9F); LC Rt 2.40 min; MS 456 (M+1).

hPRP IC₅₀=368 nM

### Example 3

### 1-{2-Methoxy-6-[2-(4-trifluoromethoxy-phenyl)-ethylamino]-pyrimidin-4-yl}-piperidine-2-carboxylic acid trifluoroacetate

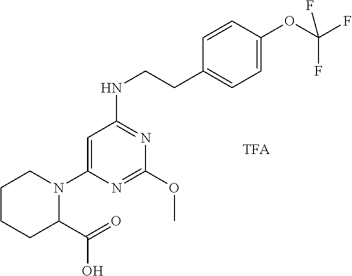 [SMILES: COc1nc(NCCc2ccc(OC(F)(F)F)cc2)cc(N2CCCCC2C(=O)O)n1]

### 3A. 1-(6-{tert-Butoxycarbonyl-[2-(4-trifluoromethoxy-phenyl)-ethyl]-amino}-2-methoxy-pyrimidin-4-yl)-piperidine-2-carboxylic acid ethyl ester

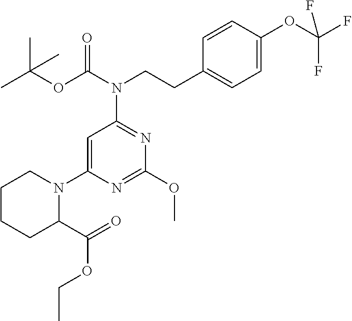 [SMILES: CCOC(=O)C1CCCCN1c1cc(N(CCc2ccc(OC(F)(F)F)cc2)C(=O)OC(C)(C)C)nc(OC)n1]

A solution of trifluoro-methanesulfonic acid 6-{tert-butoxycarbonyl-[2-(4-trifluoromethoxy-phenyl)-ethyl]-amino}-2-methoxy-pyrimidin-4-yl ester (144 mg, 0.26 mmol) and piperidine-2-carboxylic acid ethyl ester hydrochloride (99 mg, 0.51 mmol), and DIEA (89 μL, 0.51 mmol) in DMF (3 mL) was heated at 85° C. overnight. The reaction mixture was concentrated in vacuo, and the residue was partitioned between CH₂Cl₂ and water. The two layers were separated, and the organic layer was washed with 10% citric acid, saturated NaHCO₃, water, and brine, dried over Na₂SO₄, filtered, and concentrated in vacuo. The crude material was purified on silica gel with EtOAc/heptanes (5 to 10%) as eluent to afford the titled product (134 mg, 92%) as a colorless gum. ¹H NMR (300 MHz, CDCl₃) δ 7.17 (dd, J=8.5, 28.5 Hz, 4H), 6.99 (s, 1H), 5.84 (bs, 1H), 4.29-4.06 (m, 5H), 4.03-3.92 (m, 1H), 3.90 (s, 3H), 3.20 (dt, J=2.7, 12.7 Hz, 1H) 2.96 (t, J=7.4 Hz, 2H), 2.30 (d, J=13.0 Hz, 1H), 1.86-1.66 (m, 3H), 1.48 (s, 9H), 1.43-1.31 (m, 1H), 1.24 (t, J=6.9 Hz, 3H); ¹⁹F NMR (300 MHz, CDCl₃) δ-57.45 (s, 3F); LC Rt: 4.59 min; MS 569 (M+1).

### 3B. 1-{2-Methoxy-6-[2-(4-trifluoromethoxy-phenyl)-ethylamino]-pyrimidin-4-yl}-piperidine-2-carboxylic acid ethyl ester hydrochloride

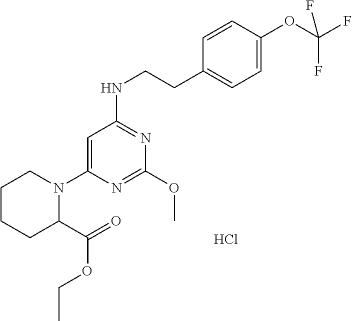 [SMILES: CCOC(=O)C1CCCCN1c1cc(NCCc2ccc(OC(F)(F)F)cc2)nc(OC)n1 ; Cl]

A solution of 1-(6-{tert-butoxycarbonyl-[2-(4-trifluoromethoxy-phenyl)-ethyl]-amino}-2-methoxy-pyrimidin-4-yl)-piperidine-2-carboxylic acid ethyl ester (120 mg, 0.21 mmol) in 4M HCl in dioxane (2 mL) was stirred at rt for 1.5 h. The reaction mixture was concentrated in vacuo to afford the titled product (93 mg, 87%) as a foam. This material was used in the next step without further purification.

### 3C. 1-{2-Methoxy-6-[2-(4-trifluoromethoxy-phenyl)-ethylamino]-pyrimidin-4-yl}-piperidine-2-carboxylic acid trifluoroacetate

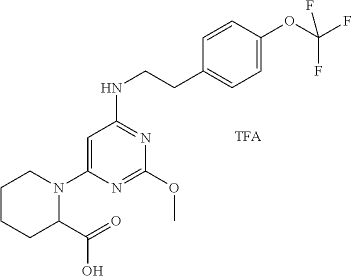 [SMILES: COc1nc(NCCc2ccc(OC(F)(F)F)cc2)cc(N2CCCCC2C(=O)O)n1 ; "TFA"]

A mixture of 1-{2-methoxy-6-[2-(4-trifluoromethoxy-phenyl)-ethylamino]-pyrimidin-4-yl}-piperidine-2-carboxylic acid ethyl ester hydrochloride (93 mg, 0.20 mmol) and NaOH (100 mg, 2.50 mmol) in MeOH (1.6 mL)/water (0.4 mL) was heated at 70° C. for 2 h. The solvent was removed in vacuo, and the residue was dissolved in water. The residue was purified by RP-HPLC to afford the titled product (87 mg, 79%) as a white solid. ¹H NMR (300 MHz, CDCl₃) δ 9.74 (bs, 1H), 7.30-7.00 (m, 4H), 5.58 (bs, 1H), 5.00 (s, 1H), 3.90 (s, 3H), 3.75-3.47 (m, 1H), 3.45-3.28 (m, 2H), 3.20 (t, J=12.8 Hz, 1H), 2.98 (t, J=6.2 Hz, 2H), 2.38 (d, J=12.1 Hz, 1H), 1.95-1.69 (m, 3H), 1.68-1.38 (m, 2H), 1.37-1.20 (m, 1H); ¹⁹F NMR (300 MHz, CDCl₃) δ-57.44 (s, 3F), −75.35 (s, 9F); LC Rt 2.97 min; MS 441 (M+1).

hPRP IC₅₀=885 nM (as HCl salt).

### Example 4

### (1-{2-Methoxy-6-[2-(4-trifluoromethoxy-phenyl)-ethylamino]-pyrimidin-4-yl}-piperidin-2-yl)-acetic acid trifluoroacetate

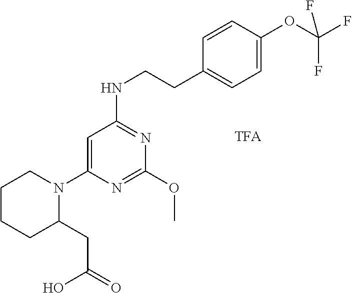 [SMILES: COc1nc(NCCc2ccc(OC(F)(F)F)cc2)cc(N2CCCCC2CC(=O)O)n1 ; "TFA"]

### 4A. [1-(6-{tert-Butoxycarbonyl-[2-(4-trifluoromethoxy-phenyl)-ethyl]-amino}-2-methoxy-pyrimidin-4-yl)-piperidin-2-yl]-acetic acid ethyl ester

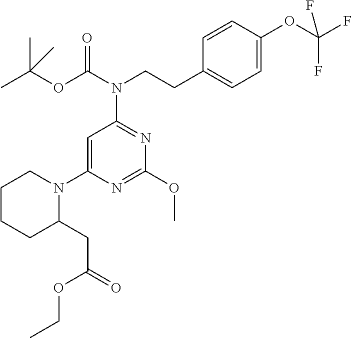 [SMILES: CCOC(=O)CC1CCCCN1c1cc(N(CCc2ccc(OC(F)(F)F)cc2)C(=O)OC(C)(C)C)nc(OC)n1]

A solution of trifluoro-methanesulfonic acid 6-{tert-butoxycarbonyl-[2-(4-trifluoromethoxy-phenyl)-ethyl]-amino}-2-methoxy-pyrimidin-4-yl ester (180 mg, 0.32 mmol) and piperidine-2-acetic acid ethyl ester hydrochloride (133 mg, 0.64 mmol) in DMF (3 mL) was stirred at rt overnight. The reaction mixture was concentrated in vacuo, and the residue was purified on silica gel with EtOAc/heptanes (10%) as eluent to afford the titled product (133 mg, 71%) as a colorless gum. ¹H NMR (300 MHz, CDCl₃) δ 7.25-7.08 (m, 4H), 6.89 (s, 1H), 4.30-4.12 (m, 3H), 4.07 (d, J=7.4 Hz, 2H), 3.92 (s, 3H), 3.03-2.86 (m, 3H), 2.62 (d, J=7.5 Hz, 2H), 1.82-1.62 (m, 5H), 1.53-1.50 (m, 2H), 1.48 (s, 9H), 1.20 (t, J=7.1 Hz, 3H); ¹⁹F NMR (300 MHz, CDCl₃) δ-57.45 (s, 3F); LC Rt: 4.53 min; MS 483 (M+1).

### 4B. (1-{2-Methoxy-6-[2-(4-trifluoromethoxy-phenyl)-ethylamino]-pyrimidin-4-yl}-piperidin-2-yl)-acetic acid ethyl ester hydrochloride

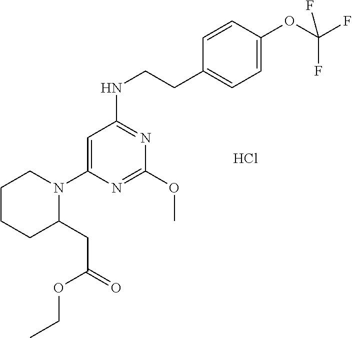 [SMILES: CCOC(=O)CC1CCCCN1c1cc(NCCc2ccc(OC(F)(F)F)cc2)nc(OC)n1 ; Cl]

A solution of [1-(6-{tert-butoxycarbonyl-[2-(4-trifluoromethoxy-phenyl)-ethyl]-amino}-2-methoxy-pyrimidin-4-yl)-piperidin-2-yl]-acetic acid ethyl ester (125 mg, 0.21 mmol) in 4M HCl in dioxane (2 mL) was stirred at rt for 1 h. The reaction mixture was concentrated in vacuo, and the residue was purified on silica gel with EtOAc/heptane (50%) and then MeOH/CH₂Cl₂ (2%) to afford the titled product (84 mg, 80%) as a foam. This material was used in the next step without further purification.

### 4C. (1-{2-Methoxy-6-[2-(4-trifluoromethoxy-phenyl)-ethylamino]-pyrimidin-4-yl}-piperidin-2-yl)-acetic acid trifluoroacetate

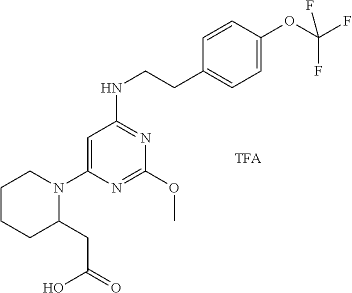 [SMILES: COc1nc(NCCc2ccc(OC(F)(F)F)cc2)cc(N2CCCCC2CC(=O)O)n1 ; "TFA"]

A mixture of (1-{2-methoxy-6-[2-(4-trifluoromethoxy-phenyl)-ethylamino]-pyrimidin-4-yl}-piperidin-2-yl)-acetic acid ethyl ester hydrochloride (84 mg, 0.16 mmol) and NaOH (100 mg, 2.50 mmol) in MeOH (1.6 mL)/water (0.4 mL) was heated at 70° C. for 2 h. The solvent was removed in vacuo, and the residue was dissolved in water. The resulting solution was purified by RP-HPLC to afford the titled product (54 mg, 55%) as a white solid. ¹H NMR (300 MHz, CDCl₃) δ 9.44 (bs, 1H), 7.30-7.07 (m, 4H), 5.87-4.17 (m, 3H), 3.92 (s, 3H), 3.80-3.43 (m, 3H), 3.41-3.24 (m, 3H), 2.94 (t, J=6.9 Hz, 3H), 2.82-2.50 (m, 2H), 1.89-1.59 (m, 4H), 1.58-1.40 (m, 1H), 1.39-1.21 (m, 1H); ¹⁹F NMR (300 MHz, CDCl₃) δ-57.43 (s, 3F), −75.30 (s, 3F); LC Rt 2.92 min; MS 455 (M+1).

hPRP IC₅₀=32 nM

### Example 5

### (2-Methoxy-6-morpholin-4-yl-pyrimidin-4-yl)-[2-(4-trifluoromethoxy-phenyl)-ethyl]-amine

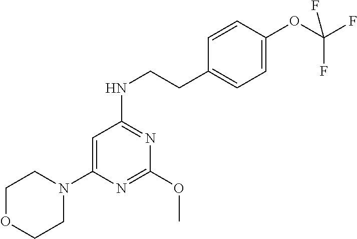 [SMILES: COc1nc(NCCc2ccc(OC(F)(F)F)cc2)cc(N2CCOCC2)n1]

A mixture of (6-chloro-2-methoxy-pyrimidin-4-yl)-[2-(4-trifluoromethoxy-phenyl)-ethyl]-amine (348 mg, 1 mmol) and morpholine (174 mg, 2 mmol) in 10 ml toluene was heated at 110° C. for 12 h. The reaction mixture was concentrated in vacuo. Purification by flash chromatography on SiO₂ eluting with 75% ethyl acetate/heptane gave 0.39 g, (97%) of the titled compound. ¹H NMR (300 MHz, CDCl₃): δ 7.2-7.1 (m, 4H), 5.1 (s, 1H), 4.6 (m, 1H), 3.9 (s, 3H), 3.7 (m, 4H), 3.5 (m, 6H), 2.9 (m, 2H). MS m/z: [M+H]⁺=399.

hPRP IC₅₀=1176 nM (as HCl salt).

### Example 6

### 4-(1-{2-Methoxy-6-[2-(4-trifluoromethoxy-phenyl)-ethylamino]-pyrimidin-4-yl}-piperazine-1-carboxylic acid ethyl ester

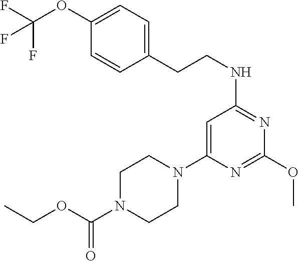 [SMILES: CCOC(=O)N1CCN(c2cc(NCCc3ccc(OC(F)(F)F)cc3)nc(OC)n2)CC1]

A mixture of ethyl piperazine carboxylate (1.06 g, 6.51 mmol), (6-chloro-2-methoxy-pyrimidin-4-yl)-[2-(4-trifluoromethoxy-phenyl)-ethyl]-amine (1.01 g, 2.91 mmol) and potassium carbonate (1.26 g, 9.09 mmol) in 1-methyl-2-pyrrolidone (70 mL) was heated at 140° C. overnight. The mixture was cooled to rt, partitioned between H₂O and EtOAc. The two layers were separated and the organic layer was washed with brine, dried over MgSO₄, filtered, and concentrated in vacuo. The crude material was purified on silica gel with heptane/EtOAc (40/60) as eluent to yield a light orange solid (1.30 g, 95%). ¹H NMR (300 MHz, CDCl₃) δ 7.25-7.22 (m, 2H), 7.16-7.14 (m, 2H), 5.11 (s, 1H), 4.75 (br s, 2H), 4.17 (q, 2H), 3.86 (s, 3H), 3.54 (br s, 6H), 2.91 (t, 2H), 2.07-2.04 (m, 3H), 1.28 (t, 3H). ¹⁹F NMR (282 MHz, CDCl₃) δ-58.4 (s, 3F). MS m/z: [M+H]⁺=470

hPRP IC₅₀=609 nM

### Example 7

### 1-{2-Methoxy-6-[2-(4-trifluoromethoxy-phenyl)-ethylamino]-pyrimidin-4-yl}-3-methyl-pyrrolidine-3-carboxylic acid

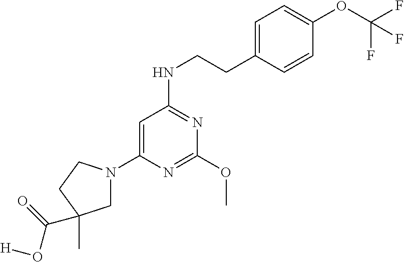 [SMILES: COc1nc(NCCc2ccc(OC(F)(F)F)cc2)cc(N2CCC(C)(C(=O)O)C2)n1]

A mixture of 3-methyl-pyrrolidine-3-carboxylic acid (0.26 g, 2 mmol), (6-chloro-2-methoxy-pyrimidin-4-yl)-[2-(4-trifluoromethoxy-phenyl)-ethyl]-amine (0.35 g, 1 mmol) and potassium carbonate (0.55 g, 4 mmol) in 1-methyl-2-pyrrolidone (5 mL) was heated at 140° C. overnight. The reaction mixture was diluted with EtOAc and was washed with H₂O and brine. The organic layer was dried over MgSO₄, filtered, and concentrated in vacuo. The crude material was purified by flash chromatography with 100% EtOAc to give the title compound (0.145 g, 33%). ¹H NMR (300 MHz, DMSO-d₆): δ 12.6 (bs, 1H), 7.4 (d, 2H), 7.3 (d, 2H), 6.7 (bs, 1H), 5.8 (s, 1H), 5.0 (s, 1H), 3.7 (s, 3H), 3.4 (m, 4H), 3.2 (m, 1H), 2.8 (m, 2H), 2.3 (m, 1H), 1.8 (m, 1H), 1.3 (s, 3H). MS m/z: [M+H]⁺=441.

hPRP IC₅₀=317 nM

### Example 8

### (3S,4S)-4-Isopropyl-1-{2-methoxy-6-[2-(4-trifluoromethoxy-phenyl)-ethylamino]-pyrimidin-4-yl}-pyrrolidine-3-carboxylic acid

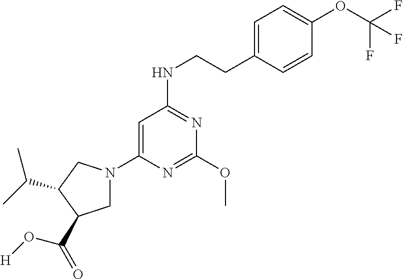 [SMILES: COc1nc(NCCc2ccc(OC(F)(F)F)cc2)cc(N2C[C@@H](C(=O)O)[C@H](C(C)C)C2)n1]

### 8A. (3S,4S)-4-Isopropyl-1-{2-methoxy-6-[2-(4-trifluoromethoxy-phenyl)-ethylamino]-pyrimidin-4-yl}-pyrrolidine-3-carboxylic acid methyl ester

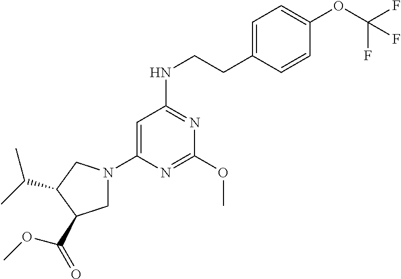 [SMILES: COC(=O)[C@@H]1CN(c2cc(NCCc3ccc(OC(F)(F)F)cc3)nc(OC)n2)C[C@H]1C(C)C]

The title compound was prepared in a similar manner as described in Example 7 using (3S,4S)-4-isopropyl-pyrrolidine-3-carboxylic acid methyl ester as the starting material. ¹H NMR (300 MHz, CDCl₃): δ 7.3-7.1 (m, 5H), 4.8 (s, 1H), 4.5 (m, 1H), 3.8 (s, 3H), 3.7 (s, 3H), 3.5 (m, 3H), 3.2 (m, 1H), 2.9 (m, 3H), 2.5 (m, 1H), 1.8 (m, 1H), 1.6 (m, 1H), 0.9 (m, 6H). MS m/z: [M+H]⁺=483

### 8B. (3S,4S)-4-Isopropyl-1-{2-methoxy-6-[2-(4-trifluoromethoxy-phenyl)-ethylamino]-pyrimidin-4-yl}-pyrrolidine-3-carboxylic acid

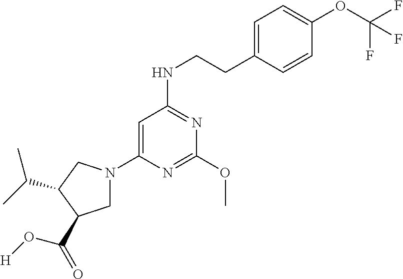 [SMILES: COc1nc(NCCc2ccc(OC(F)(F)F)cc2)cc(N2C[C@@H](C(=O)O)[C@H](C(C)C)C2)n1]

To a solution of (3S,4S)-4-isopropyl-1-{2-methoxy-6-[2-(4-trifluoromethoxy-phenyl)-ethylamino]-pyrimidin-4-yl}-pyrrolidine-3-carboxylic acid methyl ester (0.14 g, 0.29 mmol) in MeOH (15 ml) was added 1N NaOH (3 mL, 3 mmol). The reaction mixture was stirred for 1 h at rt and then 50° C. for 2 h. The mixture was then cooled to 0° C. and was acidified to pH=6 with 1N HCl. The resulting precipitate was collected to give the titled compound (0.12 g, 88%). ¹H NMR (300 MHz, DMSO-d₆): δ 12.6 (bs, 1H), 7.4 (d, 2H), 7.3 (d, 2H), 6.8 (bs, 1H), 5.1 (s, 1H), 3.8 (s, 3H), 3.7 (m, 1H), 3.4 (m, 4H), 3.1 (m, 1H), 2.8 (m, 3H), 2.3 (m, 1H), 1.7 (m, 1H), 0.9 (m, 6H). MS m/z: [M+H]⁺=469.

hPRP IC₅₀=376 nM

### Example 9

### 1-{2-Methoxy-6-[2-(4-trifluoromethoxy-phenyl)-ethylamino]-pyrimidin-4-yl}-pyrrolidine-3-carboxylic acid

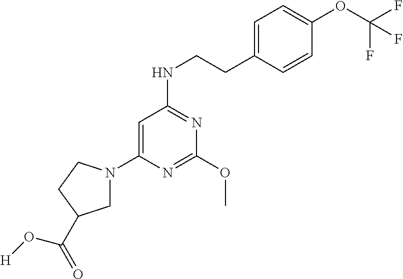 [SMILES: COc1nc(NCCc2ccc(OC(F)(F)F)cc2)cc(N2CCC(C(=O)O)C2)n1]

The title compound was prepared in a similar manner as described in Example 7 using pyrrolidine-3-carboxylic acid as the starting material. ¹H NMR (300 MHz, DMSO-d₆): δ 12.6 (bs, 1H), 7.4 (d, 2H), 7.3 (d, 2H), 6.7 (bs, 1H), 5.8 (s, 1H), 5.1 (s, 1H), 3.7 (s, 3H), 3.6-3.3 (m, 5H), 3.1 (m, 1H), 2.8 (m, 2H), 2.1 (m, 2H). MS m/z: [M+H]⁺=427.

hPRP IC₅₀=575 nM

### Example 10

### (3R,4S)-1-{2-Methoxy-6-[2-(4-trifluoromethoxy-phenyl)-ethylamino]-pyrimidin-4-yl}-4-phenyl-pyrrolidine-3-carboxylic acid

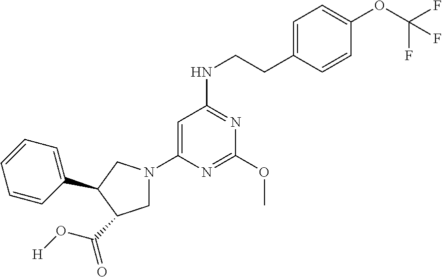 [SMILES: COc1nc(NCCc2ccc(OC(F)(F)F)cc2)cc(N2C[C@H](C(=O)O)[C@@H](c3ccccc3)C2)n1]

### 10A. (3R,4S)-1-{2-Methoxy-6-[2-(4-trifluoromethoxy-phenyl)-ethylamino]-pyrimidin-4-yl}-4-phenyl-pyrrolidine-3-carboxylic acid methyl ester

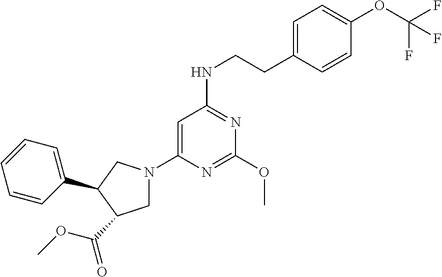 [SMILES: COC(=O)[C@H]1CN(c2cc(NCCc3ccc(OC(F)(F)F)cc3)nc(OC)n2)C[C@@H]1c1ccccc1]

The title compound was prepared in a similar manner as described in Example 7 using (3R,4S)-4-phenyl-pyrrolidine-3-carboxylic acid methyl ester as the starting material. ¹H NMR (300 MHz, CDCl₃): δ 7.4-7.1 (m, 10H), 4.9 (s, 1H), 4.5 (m, 1H), 3.8 (s, 3H), 3.7 (m, 2H), 3.6 (s, 3H), 3.5 (m, 3H), 3.3 (m, 1H), 2.9 (m, 2H). MS m/z: [M+H]⁺=517.

### 10B. (3R,4S)-1-{2-Methoxy-6-[2-(4-trifluoromethoxy-phenyl)-ethylamino]-pyrimidin-4-yl}-4-phenyl-pyrrolidine-3-carboxylic acid

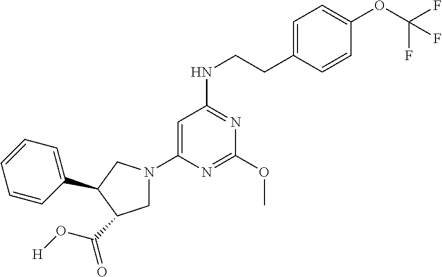 [SMILES: COc1nc(NCCc2ccc(OC(F)(F)F)cc2)cc(N2C[C@H](C(=O)O)[C@@H](c3ccccc3)C2)n1]

The title compound was prepared in a similar manner as described in Example 7 using (3R,4S)-1-{2-methoxy-6-[2-(4-trifluoromethoxy-phenyl)-ethylamino]-pyrimidin-4-yl}-4-phenyl-pyrrolidine-3-carboxylic acid methyl ester as the starting material. ¹H NMR (300 MHz, DMSO-d₆): δ 12.6 (bs, 1H), 7.4-7.2 (m, 10H), 7.0 (bs, 1H), 5.2 (s, 1H), 3.9 (m, 1H), 3.8 (s, 3H), 3.6 (m, 2H), 3.4 (m, 4H), 2.8 (m, 2H). MS m/z: [M+H]⁺=503.

hPRP IC₅₀=28 nM

### Example 11

### (3R,4S)-4-(4-Methoxy-phenyl)-1-{2-methoxy-6-[2-(4-trifluoromethoxy-phenyl)-ethylamino]-pyrimidin-4-yl}-pyrrolidine-3-carboxylic acid

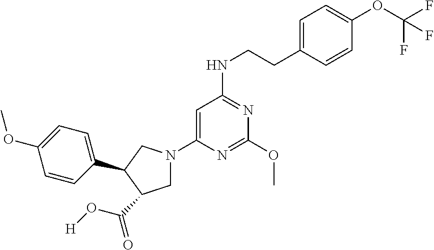 [SMILES: COc1ccc([C@H]2CN(c3cc(NCCc4ccc(OC(F)(F)F)cc4)nc(OC)n3)C[C@@H]2C(=O)O)cc1]

### 11A. (3R,4S)-4-(4-Methoxy-phenyl)-1-{2-methoxy-6-[2-(4-trifluoromethoxy-phenyl)-ethylamino]-pyrimidin-4-yl}-pyrrolidine-3-carboxylic acid methyl ester

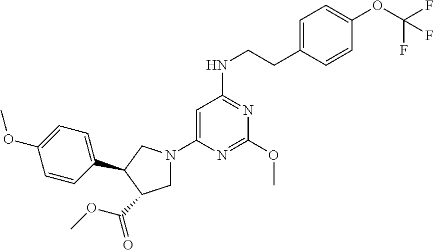 [SMILES: COC(=O)[C@H]1CN(c2cc(NCCc3ccc(OC(F)(F)F)cc3)nc(OC)n2)C[C@@H]1c1ccc(OC)cc1]

The title compound is prepared in a similar manner as in Example 7 using (3R,4S)-4-(4-methoxy-phenyl)-pyrrolidine-3-carboxylic acid methyl ester as the starting material. ¹H NMR (300 MHz, CDCl₃): δ 7.3-7.1 (m, 6H), 6.9 (d, 2H), 4.9 (s, 1H), 4.6 (m, 1H), 4.0 (m, 2H), 3.85 (s, 3H), 3.8 (s, 3H), 3.7 (m, 2H), 3.6 (s, 3H), 3.5 (m, 3H), 3.2 (m, 1H), 2.9 (m, 2H). MS m/z: [M+H]⁺=547

### 11B. (3R,4S)-4-(4-Methoxy-phenyl)-1-{2-methoxy-6-[2-(4-trifluoromethoxy-phenyl)-ethylamino]-pyrimidin-4-yl}-pyrrolidine-3-carboxylic acid

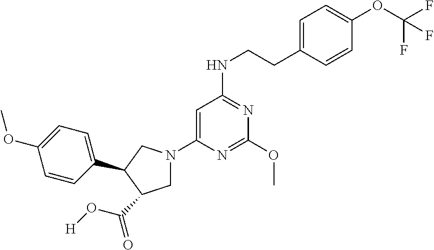 [SMILES: COc1ccc([C@H]2CN(c3cc(NCCc4ccc(OC(F)(F)F)cc4)nc(OC)n3)C[C@@H]2C(=O)O)cc1]

The title compound was prepared in a similar manner as described in Example 7 using (3R,4S)-4-(4-methoxy-phenyl)-1-{2-methoxy-6-[2-(4-trifluoromethoxy-phenyl)-ethylamino]-pyrimidin-4-yl}-pyrrolidine-3-carboxylic acid methyl ester as the starting material. ¹H NMR (300 MHz, DMSO-d₆): δ 12.5 (bs, 1H), 7.4-7.2 (m, 7H), 6.9 (d, 2H), 6.7 (bs, 1H), 5.1 (s, 1H), 3.9 (m, 2H), 3.7 (s, 3H), 3.6-3.4 (m, 3H), 3.3 (s, 3H), 3.2 (m, 2H), 2.9 (m, 2H). MS m/z: [M+H]⁺=533.

hPRP IC₅₀=409 nM

### Example 12

### [6-(3,4-Dihydro-1H-isoquinolin-2-yl)-2-methoxy-pyrimidin-4-yl]-[2-(4-trifluoromethoxy-phenyl)-ethyl]-amine trifluoroacetic acid salt

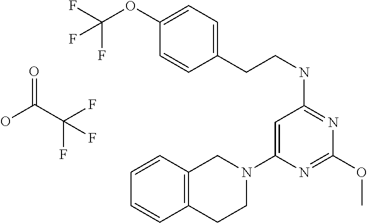 [SMILES: O=C(O)C(F)(F)F ; COc1nc(NCCc2ccc(OC(F)(F)F)cc2)cc(N2CCc3ccccc3C2)n1]

The title compound was prepared in a similar manner as described in Example 7 using 1,2,3,4-tetrahydroisoquinoline as the starting material. ¹H NMR (300 MHz, DMSO-d₆) δ 7.56 (br s, 1H), 7.40 (d, 2H), 7.29 (d, 2H), 7.24-7.21 (m, 4H), 5.53 (s, 1H), 4.73 (s, 2H), 3.93 (s, 3H), 3.82 (br s, 2H), 3.52 (br s, 2H), 2.92-2.87 (m, 4H). ¹⁹F NMR (282 MHz, DMSO-d₆) δ-57.5 (s, 3F), −74.8 (s, 3F). MS m/z: [M+H]⁺=445

hPRP IC₅₀=1220 nM

### Example 13

### [2-Methoxy-6-(4-phenyl-piperazin-1-yl)-pyrimidin-4-yl]-[2-(4-trifluoromethoxy-phenyl)-ethyl]-amine

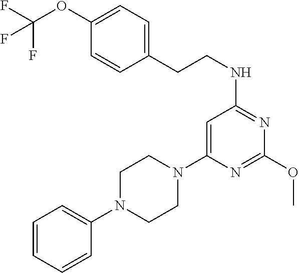 [SMILES: COc1nc(NCCc2ccc(OC(F)(F)F)cc2)cc(N2CCN(c3ccccc3)CC2)n1]

The title compound was prepared in a similar manner as described in Example 7 using 1-phenylpiperazine as the starting material. ¹H NMR (300 MHz, DMSO-d₆) δ 7.38 (d, 2H), 7.28 (d, 2H), 7.22 (d, 2H), 6.98 (d, 2H), 6.83-6.78 (m, 2H), 5.37 (s, 1H), 3.75 (s, 3H), 3.58 (br s, 4H), 3.45-3.43 (m, 2H), 3.18 (br s, 4H), 2.85 (t, 2H). ¹⁹F NMR (282 MHz, DMSO-d₆) δ-57.2 (s, 3F). MS m/z: [M+H]⁺=474.

hPRP IC₅₀=397 nM

### Example 14

### ((R)-1-{2-Methoxy-6-[2-(4-trifluoromethoxy-phenyl)-ethylamino]-pyrimidin-4-yl}-pyrrolidin-3-yl)-acetic acid

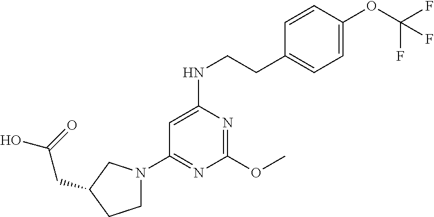 [SMILES: COc1nc(NCCc2ccc(OC(F)(F)F)cc2)cc(N2CC[C@H](CC(=O)O)C2)n1]

### 14A. [(R)-1-(6-{tert-Butoxycarbonyl-[2-(4-trifluoromethoxy-phenyl)-ethyl]-amino}-2-methoxy-pyrimidin-4-yl)-pyrrolidin-3-yl]-acetic acid

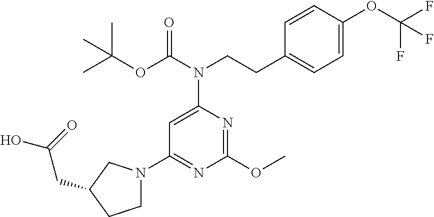 [SMILES: COc1nc(N2CC[C@H](CC(=O)O)C2)cc(N(CCc2ccc(OC(F)(F)F)cc2)C(=O)OC(C)(C)C)n1]

Trifluoro-methanesulfonic acid 6-{tert-butoxycarbonyl-[2-(4-trifluoromethoxy-phenyl)-ethyl]-amino}-2-methoxy-pyrimidin-4-yl ester (213 mg, 0.38 mmol) and pyrrolidin-3-(R)-yl-acetic acid (126 mg, 0.76 mmol) were heated at 85° C. in DMF (6 mL) for 5 h. The reaction mixture was cooled to rt then concentrated in vacuo. The residue was taken up in CH₂Cl₂ and chromatographed using 5% MeOH/CH₂Cl₂ as the eluent to provide the product (200 mg, 98%) as a white solid. ¹H NMR (300 MHz, CDCl₃) δ 7.21 (d, 2H), 7.17 (d, 2H), 6.61 (s, 1H), 4.20 (t, 2H), 3.97 (s, 3H), 2.99 (t, 2H), 2.98 (s, 2H), 2.90 (s, 2H), 2.75-2.65 (m, 1H), 2.50 (d, 2H), 2.27-2.20 (m, 1H), 1.78-1.65 (m, 1H), 1.42 (s, 9H); MS m/z: [M+H]⁺=541.

### 14B. ((R)-1-{2-Methoxy-6-[2-(4-trifluoromethoxy-phenyl-ethylamino]-pyrimidin-4-yl}-pyrrolidin-3-yl)-acetic acid

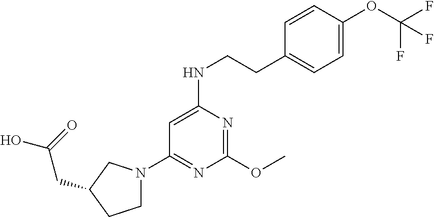 [SMILES: COc1nc(NCCc2ccc(OC(F)(F)F)cc2)cc(N2CC[C@H](CC(=O)O)C2)n1]

[(R)-1-(6-{tert-Butoxycarbonyl-[2-(4-trifluoromethoxy-phenyl)-ethyl]-amino}-2-methoxy-pyrimidin-4-yl)-pyrrolidin-3-yl]-acetic acid (200 mg, 0.37 mmol) in Et₂O (2 mL) was treated with 2M HCl in ether (2 mL, 4 mmol) and heated at 30° C. for 4.5 h. The reaction mixture was concentrated to dryness in vacuo and the residue purified by reverse phase HPLC using 10-70% CH₃CN/(0.1% TFA/water) as the eluent. The fraction collected at 62% CH₃CN/(0.1% TFA/water) was lyophilized to afford the titled product as a white solid (10 mg, 6%). ¹H NMR (300 MHz, DMSO-d₆) δ 7.38 (d, 2H), 7.29 (d, 2H), 5.16 (s, 1H), 3.90 (s, 3H), 3.70-3.60 (br m, 2H), 3.19-3.00 (br m, 2H), 2.87 (t, 2H), 2.58-2.53 (m, 1H), 2.40 (d, 2H), 2.05-2.18 (m, 2H), 1.70-1.60 (m, 2H); MS m/z: [M+H]⁺=441.

hPRP IC₅₀=713 nM

### Example 15

### (±)-trans-4-(2-Methoxy-phenyl)-1-{2-methoxy-6-[2-(4-trifluoromethoxy-phenyl)-ethylamino]-pyrimidin-4-yl}-pyrrolidine-3-carboxylic acid

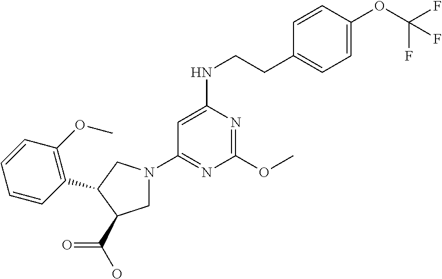 [SMILES: COc1nc(NCCc2ccc(OC(F)(F)F)cc2)cc(N2C[C@@H](C(=O)O)[C@H](c3ccccc3OC)C2)n1]

### 15A. 1-(6-{tert-Butoxycarbonyl-[2-(4-trifluoromethoxy-phenyl)-ethyl]-amino}-2-methoxy-pyrimidin-4-yl)-(±)-trans-4-(2-methoxy-phenyl)-pyrrolidine-3-carboxylic acid

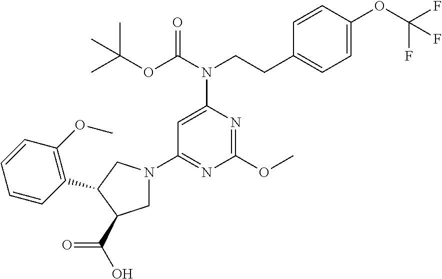 [SMILES: COc1nc(N2C[C@@H](C(=O)O)[C@H](c3ccccc3OC)C2)cc(N(CCc2ccc(OC(F)(F)F)cc2)C(=O)OC(C)(C)C)n1]

Trifluoro-methanesulfonic acid 6-{tert-butoxycarbonyl-[2-(4-trifluoromethoxy-phenyl)-ethyl]-amino}-2-methoxy-pyrimidin-4-yl ester (111 mg, 0.20 mmol), (±)-trans-4-(2-methoxy-phenyl)-pyrrolidine-3-carboxylic acid hydrochloride (102 mg, 0.40 mmol) and iPr₂EtN (0.07 mL, 0.41 mmol) were heated at 85° C. in DMF (4 mL) overnight. The reaction mixture was cooled to rt and concentrated in vacuo. The residue was purified by SiO₂ column chromatography using 5% MeOH/CH₂Cl₂ as the eluent to provide the product (106 mg, 85%) as a white solid. ¹H NMR (300 MHz, CDCl₃) δ 7.20-7.11 (m, 6H), 6.92-6.88 (m, 2H), 6.71 (s, 1H), 4.19 (t, 2H), 4.13-4.03 (m, 1H), 3.94 (s, 3H), 3.84 (s, 3H), 3.52-3.45 (m, 4H), 2.96 (t, 2H), 1.47 (s, 9H); MS m/z: [M+H]⁺=633.

### 15B. (±)-trans-4-(2-Methoxy-phenyl)-1-{2-methoxy-6-[2-(4-trifluoromethoxy-phenyl)-ethylamino]-pyrimidin-4-yl}-pyrrolidine-3-carboxylic acid

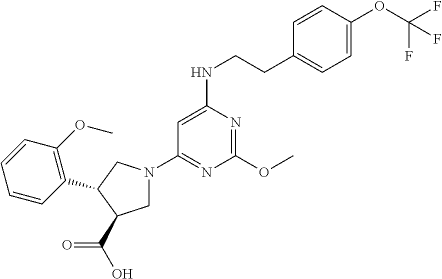 [SMILES: COc1nc(NCCc2ccc(OC(F)(F)F)cc2)cc(N2C[C@@H](C(=O)O)[C@H](c3ccccc3OC)C2)n1]

1-(6-{tert-Butoxycarbonyl-[2-(4-trifluoromethoxy-phenyl)-ethyl]-amino}-2-methoxy-pyrimidin-4-yl)-(±)-trans-4-(2-methoxy-phenyl)-pyrrolidine-3-carboxylic acid (106 mg, 0.17 mmol) in CH₂Cl₂ (5 mL) was treated with TFA (2 mL) and stirred at rt for 5.5 h. The reaction mixture was concentrated to dryness in vacuo and purified by SiO₂ column chromatography using 5% MeOH/CH₂Cl₂ as the eluent to provide the product (80 mg, 91%) as an off-white solid. ¹H NMR (300 MHz, DMSO-d₆) δ 12.60 (br s, 1H), 7.58-7.38 (m, 2H), 7.29-7.27 (m, 4H), 7.01-6.92 (m, 2H), 5.29 (s, 1H), 4.00-3.68 (m, 4H), 3.92 (s, 3H), 3.81 (s, 3H), 3.60-3.40 (m, 4H), 2.90-2.86 (b, 2H); MS m/z: [M+H]⁺=533.

hPRP IC₅₀=436 nM

### PHARMACOLOGICAL TESTING

Assessment of Antagonist Activities of Compounds on BW245C-induced cAMP Accumulation in Human Platelet Rich Plasma (hPRP) by HTRF cAMP Assay

The purpose of the assay is to assess compound antagonist activity at the human prostaglandin D₂ receptor (DP) (DP1) in the presence of plasma proteins. DP is a Gs-protein coupled receptor, the activation of which induces cAMP accumulation. BW245C is a DP selective agonist. Therefore, by measuring inhibition of BW245C-induced 3′-5′-cyclic adenosine monophosphate (cAMP) accumulation in human platelet-rich plasma (PRP), the assay enables us to identify or confirm antagonist compounds at the human DP and/or IP receptors.

The principle of the assay is based on HTRF technology (Homogeneous Time-Resolved Fluorescence). The method is a competitive immunoassay between native cAMP produced by cells and the tracer cAMP labeled with the dye d2. The tracer is visualized by a monoclonal antibody anti-cAMP labeled with cryptate. The specific signal (i.e. energy transfer) is inversely related to the concentration of cAMP in the standards or samples. The assay was carried out using the cAMP HiRange HTRF kit from Cisbio (catalog number 62AM6PEB, 888-963-4567).

Preparation of Human Platelet Rich Plasma (hPRP): Human blood was obtained from sanofi-aventis on-site blood donor panel. The blood was gently transferred from the blood bag into a 50 ml centrifuge tube and centrifuged at 223×g (1000 rpm) for 15 minutes without break. The top layer (PRP) was aspirated slowly and transferred to a 250 ml centrifuge tube. The PRP was placed in the cell culture hood for approximately 30 minutes before use.

Preparation of IBMX: IBMX is a phosphodiesterase (PDE) inhibitor and is included in the assay to prevent breakdown of cAMP. 1M IBMX stock was prepared in DMSO. 20 μL of 1M IBMX stock was then added into 30 μL of DMSO to obtain a 400 mM IBMX DMSO solution. This was further diluted 1:50 in 0.9% sodium chloride to obtain an 8 mM IBMX working solution. The solution was sonicated for 60 minutes before use.

Preparation of BW245C: 10 mM BW245C stock was prepared in DMSO and aliquots were stored at −80° C. On the day of the assay, 10 mM BW245C stock was diluted 1 to 400 in DMSO to make a 25 μM solution. 100 μL of the 25 μM BW245C solution was added to 4900 μL of 0.9% sodium chloride to make a 500 nM working solution.

Dilution of compounds: 10 mM compound DMSO stock solutions were serially diluted 1:3 in DMSO in a 96-well plate to achieve 11 different concentrations ranging from 10 mM to 0.00017 mM. A further 1:20 dilution in 0.9% sodium chloride solution was carried out for each concentration to obtain working concentrations ranging from 500 μM to 0.0085 μM (11 points) for each compound. For positive and negative controls, DMSO (without compound) was diluted 1:20 in 0.9% sodium chloride solution.

Preparation of cAMP standards, cAMP-d2 and anti-cAMP cryptate (all in the assay kit): cAMP standard was reconstituted by adding distilled water according to the manufacturer's instruction (456 μL of water usually). The reconstituted cAMP standard was serially diluted 1:4 in 0.9% sodium chloride solution to achieve 11 different concentrations. cAMP-d2 was reconstituted by adding 2 mL of distilled water and then further diluting it in 8 mL of lysis buffer (in the kit). Anti-cAMP cryptate was reconstituted by adding 1.1 mL of distilled water and then further diluting it in 4.4 mL of lysis buffer.

Assay Procedure: In the assay, each compound was run in duplicate. The final assay volume was 50 μl in each well.

In the assay plate, 42 μL of platelet rich plasma (PRP) was added in each well. This was followed by the addition in each well of 2.5 μL of 8 mM IBMX (final concentration 400 μM) and 3 μL of diluted compound at varying concentrations (final concentrations ranging from 30,000 nM to 0.51 nM, 11 points for each compound). In the positive and negative control wells, 3 μL of diluted DMSO solution was added instead of compound. The plate was tapped gently and incubated at 37° C. for 20 minutes. This was followed by the addition of 2.5 μL of 500 nM BW245C (final concentration 25 nM), or in the negative control wells, 2.5 μL diluted DMSO solution. The assay plate was further incubated for 20 minutes at room temperature without shaking

In a separate plate for the cAMP standards, 25 μL of PRP was added to each well. This was followed by the addition in each well of 25 μL of the diluted cAMP standard at varying concentrations (final concentrations ranging from 2800 nM to 0.0027 nM, 11 points in duplicate).

For detecting cAMP, 25 μL of cAMP-d2 and then 25 μl of anti-cAMP cryptate were added to each well in the assay plate and in the plate containing the cAMP standard. The plates were incubated at room temperature for at least 1 hour without shaking (the signals will be stable for at least 24 hours) before reading on a compatible HTRF reader—LGL analyst AD. The fluorescence counts at 665 nm and 620 nm were recorded and the ratio of 665 nm/620 nm was calculated.

### Data Analysis:

cAMP standard curve was generated using nonlinear regression (curve fit) in Graphpad Prism version 4.03 (X axis: log [cAMP] (M) from cAMP standards; Y axis: ratio 665 nm/620 nm*10000 from the LGL analyst). The individual 665 nm/620 nm*10000 data from each sample well were then calculated in Graphpad Prism version 4.03 against the standard curve to obtain cAMP concentration in each well.

The cAMP concentrations in positive control wells (i.e. BW245C only without compound) were averaged and used to normalize the values from all other wells:

% BW245C-induced cAMP accumulation=(cAMP concentration in individual well/average cAMP concentration in positive control wells)*100.

Concentration response curves for each compound were generated using nonlinear regression (curve fit) in Graphpad Prism version 4.03. (X is the logarithm of compound concentrations; Y is % BW245C-induced cAMP accumulation). Equation for nonlinear regression—sigmoidal dose-response with variable slope is:

Y=Bottom+(Top-Bottom)/(1+10̂((Log EC50-X)*HillSlope)).

We claim:

1. A compound of formula (I) 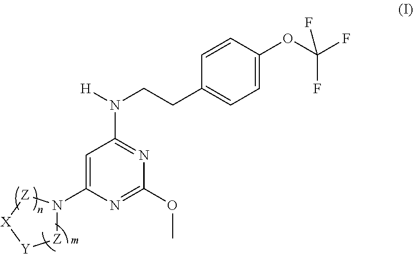
wherein m and n, independently of each other, are selected from the integers 0, 1, 2 or 3;
X and Y, independently of each other, are selected from CR₁R₂, NR₁ or O, wherein X and Y cannot both be O;
or X and Y, taken together with the bond between them, form a phenyl group optionally substituted by one to four R₃ groups;
each Z, independently of each other, is CR₁R₂;
R₁, R₂ and R₃, independently of each other, are selected from the group consisting of H, halogen, aryl, amino, optionally substituted alkyl, optionally substituted alkoxy, and carboxy;
wherein optionally substituted alkyl, may be substituted by one to three of the same or different of halogen, carboxy, cyano, hydroxy, amino or aryl;
wherein optionally substituted alkoxy, may be substituted by one to three of the same or different of halogen, carboxy, cyano, amino or aryl;
wherein each aryl moiety, independently of each other, may be optionally substituted by hydroxy, amino, alkyl, alkoxy, carboxy or alkoxycarbonyl;
provided that a halogen cannot be bonded to an N; and
provided the compound is not (1-{2-methoxy-6-[2-(4-trifluoromethoxy-phenyl)-ethylamino]-pyrimidin-4-yl}-piperidin-3-yl)-acetic acid;
or an enantiomer thereof, or a pharmaceutically acceptable salt thereof.

2. A compound according to claim 1 that is selected from the group consisting of:
4-{2-methoxy-6-[2-(4-trifluoromethoxy-phenyl)-ethylamino]-pyrimidin-4-yl}morpholine-2-carboxylic acid,
(4-{2-methoxy-6-[2-(4-trifluoromethoxy-phenyl)-ethylamino]-pyrimidin-4-yl}-piperazin-1-yl)-acetic acid,
1-{2-methoxy-6-[2-(4-trifluoromethoxy-phenyl)-ethylamino]-pyrimidin-4-yl}-piperidine-2-carboxylic acid,
(1-{2-methoxy-6-[2-(4-trifluoromethoxy-phenyl)-ethylamino]-pyrimidin-4-yl}-piperidin-2-yl)-acetic acid,
(2-methoxy-6-morpholin-4-yl-pyrimidin-4-yl)-[2-(4-trifluoromethoxy-phenyl)-ethyl]-amine,
4-(1-{2-methoxy-6-[2-(4-trifluoromethoxy-phenyl)-ethylamino]-pyrimidin-4-yl}-piperazine-1-carboxylic acid ethyl ester,
1-{2-methoxy-6-[2-(4-trifluoromethoxy-phenyl)-ethylamino]-pyrimidin-4-yl}-3-methyl-pyrrolidine-3-carboxylic acid,
(3S,4S)-4-isopropyl-1-{2-methoxy-6-[2-(4-trifluoromethoxy-phenyl)-ethylamino]-pyrimidin-4-yl}-pyrrolidine-3-carboxylic acid,
1-{2-methoxy-6-[2-(4-trifluoromethoxy-phenyl)-ethylamino]-pyrimidin-4-yl}-pyrrolidine-3-carboxylic acid,
(3R,4S)-1-{2-methoxy-6-[2-(4-trifluoromethoxy-phenyl)-ethylamino]-pyrimidin-4-yl}-4-phenyl-pyrrolidine-3-carboxylic acid,
(3R,4S)-4-(4-methoxy-phenyl)-1-{2-methoxy-6-[2-(4-trifluoromethoxy-phenyl)-ethylamino]-pyrimidin-4-yl}-pyrrolidine-3-carboxylic acid,
[6-(3,4-dihydro-1H-isoquinolin-2-yl)-2-methoxy-pyrimidin-4-yl]-[2-(4-trifluoromethoxy-phenyl)-ethyl]-amine,
[2-methoxy-6-(4-phenyl-piperazin-1-yl)-pyrimidin-4-yl]-[2-(4-trifluoromethoxy-phenyl)-ethyl]-amine,
((R)-1-{2-methoxy-6-[2-(4-trifluoromethoxy-phenyl)-ethylamino]-pyrimidin-4-yl}-pyrrolidin-3-yl)-acetic acid, and
(±)-trans-4-(2-methoxy-phenyl)-1-{2-methoxy-6-[2-(4-trifluoromethoxy-phenyl)-ethylamino]-pyrimidin-4-yl}-pyrrolidine-3-carboxylic acid.

3. A pharmaceutical composition comprising a pharmaceutically effective dose of a compound according to claim 1 in admixture with a pharmaceutically acceptable carrier.

4. A method of treating a patient suffering from an allergic disorder, bronchial asthma, allergic rhinitis, allergic dermatitis, macular degeneration, wet macular degeneration, dry macular degeneration, allergic conjunctivitis, or chronic obstructive pulmonary, comprising administering thereto a pharmaceutically effective amount of the compound according to claim 1.

5. A pharmaceutical composition comprising a pharmaceutically effective amount of a compound according to claim 1 and a compound selected from the group consisting of an antihistamine, a leukotriene antagonist, a beta agonist, a PDE4 inhibitor, a TP antagonist and a CrTh2 antagonist, in admixture with a pharmaceutically acceptable carrier.

6. A pharmaceutical composition according to claim 5, wherein the antihistamine is fexofenadine, loratadine, cetirizine or levocetirizine; the leukotriene antagonist is montelukast or zafirlukast; the beta agonist is albuterol, salbuterol or terbutaline; the PDE4 inhibitor is roflumilast or cilomilast; the TP antagonist is ramatroban; and the CrTh2 antagonist is ramatroban.

7. A pharmaceutical composition comprising a compound according to claim 1 and niacin, or a pharmaceutically acceptable salt thereof, or a nicotinic acid receptor agonist.

8. A pharmaceutical composition comprising a compound according to claim 1 and niacin, or a pharmaceutically acceptable salt thereof, or a nicotinic acid receptor agonist, and a statin.

9. A method of treating atherosclerosis, dyslipidemia, diabetes or a related condition while reducing substantial flushing in a patient in need thereof, comprising administering to the patient a pharmaceutical composition according to claim 7.

10. A method of treating atherosclerosis, dyslipidemia, diabetes or a related condition while reducing substantial flushing in a patient in need thereof, comprising administering to the patient a pharmaceutical composition according to claim 8.
